(19) United States
(12) Patent Application Publication
(10) Pub. No.: US 2011/0159515 A1
(43) Pub. Date: Jun. 30, 2011

(54) COMPOSITIONS AND METHODS FOR THE RAPID GROWTH AND DETECTION OF MICROORGANISMS

(75) Inventor: William Stimson, Glasgow (GB)

(73) Assignee: SOLUS SCIENTIFIC SOLUTIONS LIMITED, Glasgow (GB)

(21) Appl. No.: 12/737,995

(22) PCT Filed: Sep. 10, 2009
(86) PCT No.: PCT/GB2009/051161

§ 371 Date: Mar. 8, 2011

(30) Foreign Application Priority Data
Sep. 10, 2008 (GB) 0816559.9

(51) Int. Cl. G01N 33/569; C12N 1/00; C12N 1/20; G01N 33/84; G01N 33/64; G01N 21/76

(52) U.S. Cl. 435/7.1; 435/243; 435/252.8; 435/252.1

(57) ABSTRACT

The invention relates to assay methods for use in detecting specific materials such as core oligosaccharides derived from microorganisms, particularly pathogenic microorganisms, in a test sample. The invention further relates to compositions and methods for the rapid growth of such microorganisms enabling detection of same significantly earlier than is currently possible. In particular embodiments the invention is directed towards the rapid growth and/or detection of Salmonella, Shigella or Listeria.

### FIELD OF INVENTION

The invention relates to assay methods for use in detecting specific materials derived from microorganisms, particularly pathogenic microorganisms, in a test sample. The invention further relates to compositions and methods for the rapid growth of such microorganisms enabling detection of same significantly earlier than is currently possible.

### BACKGROUND OF INVENTION

Because food products are biological in nature they are capable of supporting the growth of a variety of contaminating microorganisms. In the United States, an estimated 76 million cases of foodborne illness occurs each year costing between $6.5 and $34.9 billion dollars in medical care and lost productivity (Buzby and Roberts, 1997; Mead et al, 1999). In Europe it has been estimated that the economic and health care costs of Salmonella are between 620 million and 3 billion Euro (David Byrne, European Commissioner for health and consumer protection, 2000).

Salmonella, Listeria, Campylobacter, Escherichia coli O157:H7 and Shigella are responsible for the majority of cases of foodborne illness. For example, Salmonella and Listeria alone were responsible for 31% and 28% respectively of food-related deaths (Mead et al, 1999) and in Japan, salmonellosis accounted for over 14% of the total foodborne illness outbreaks between 1981 and 1995 (Lee et al, 2001). In fact it has been estimated that bacteria are the causative agents of as much as 60% of the cases of foodborne illness requiring hospitalisation. As a result, one of the biggest contributors to waste is delay caused by inefficient and slow testing of products for microbial contamination. With current testing methods, manufacturers must wait from three to seven days for the results of microbial incubation. The costs arising from such delays are significant—reducing supply chain efficiency, tying up inventory and increasing spoilage.

The costs of inadequate or insufficient testing can be as, if not more, costly. For example, in 1999, it cost Sara Lee an estimated $76 million in costs related to the recall of 35 million pounds of hot dogs and deli meats at its Bil Mar Foods unit, after the food was linked to an outbreak of L.isteria According to ‘The Scotsman’, contamination of chocolate with Salmonella in 2006 cost Cadbury Schweppes an estimated £20 million in recall costs, advertising, lost revenue and subsequent improvements to its manufacturing operation. More recently in 2009, the Peanut Corporation of America, a company with an estimated $25 million in sales in 2008, filed for bankruptcy after being identified as the source of a major Salmonella outbreak in peanuts in the USA.

Therefore, detection of the presence of pathogenic microorganisms such as Salmonella, Shigella and Listeria in food, feed and environmental samples is of great economic importance. However, conventional culture methods for detection of such microorganisms are both labour intensive and time-consuming. Often such methods rely on standard processes that have been in use for more than 50 years.

In addition, pathogenic microorganisms can persist for long periods in an environment in a heavily stressed state known as ‘viable but not culturable (VNC)’ or ‘not immediately culturable (NIC)’. Such heavily stressed microorganisms show only a weak metabolic activity, often at the limits of detection, and they lose the ability to form colonies on non-selective plating media or to grow in non-selective broth media (Reissbrodt et al, (2002). However, when such nonculturable colonies exist in food and animal feed, they may still be capable of causing disease if ingested. This poses particular problems with regard to detection since such stressed microorganisms may not be revived sufficiently to be detected.

As a result, additional cell culture steps are often included in any diagnostic with the aim of reviving such cells prior to further culture, plating and detection. Hence, pre-enrichment in non-selective culture media is an essential element of conventional methods (Stephens et al, 2000). For example, the detection of Salmonella requires several stages of culture spread over as many as five days; enrichment steps are often included in the analysis to revive ‘sick’ bacteria and detection is often limited by the performance of such enrichment broths and cultures.

Thus, for the recovery of microorganisms from clinical specimens, food and other products that potentially harbour a heterogenous population of bacteria, three general types of culture media are available: (1) non-selective media for primary isolation, (2) enrichment broths and (3) selective and/or differential agars.

The formulas for such media are generally complex and include ingredients that not only inhibit growth of certain bacterial species, i.e. they are selective, but also detect several biochemical characteristics that are important in making a preliminary identification of the micro-organisms present in the specimen, i.e. they are differentiating. In order to make rational selections, microbiologists must know the composition of each formula and the purpose and relative concentration of each chemical compound included. Unfortunately the media available are often overly complex and the effect and amounts of the various components are generally little understood. Often the medium that is used is the same as that which has been used for several decades and may originally have been developed for an entirely different organism. For example, because of these inefficiencies, current detection rates of Salmonella are less than 50% within 15 days and 90% within 28 days (King, 2009).

Hence, there is a need for culture media that are well defined, do not contain surplus ingredients that may have little to no or even negative effects and are optimal for the growth and rapid culture of even stressed microorganisms. Such culture media should negate the need for secondary/additional culture steps. There is also a need for new and better detection methods that enable the isolation and/or identification of pathogenic microorganisms found in very low numbers and in a heterogenous microflora environment. Further, any such methods should be equally applicable to detection of microorganisms from a wide variety of sources such as cosmetics, food products including frozen, lyophilised and liquid products, clinical samples such as urine, stool or blood samples and environmental samples.

### SUMMARY OF THE INVENTION

In a first aspect of the invention there is provided a culture medium for the growth of at least one microorganism consisting essentially of:

(i) A base broth;

(ii) At least one growth inhibitor selected from the group consisting of brilliant green, nalidixic acid and lithium chloride; and

(iii) Optionally, at least one growth promoter selected from the group consisting of sodium tetrathionate, potassium tetrathionate, ammonium ferric citrate and sodium citrate.

For the avoidance of doubt, the term ‘consisting essentially’ as used herein includes the specified materials or steps only and additional components or elements to the extent that they do not materially affect the basic and novel characteristics of the invention.

Media can be classified as simple, complex or defined. Base broths or basal media are basically simple media that support bacteria with minimal additional components. Generally such base broths simply need to provide a source of energy and maintain correct osmolarity. Peptone, tryptone, nutrient broth (peptone, meat extract, optionally yeast extract and sodium chloride), L-broth (tryptone, yeast extract and sodium chloride), gram negative broth, tryptic soy broth, tryptic soy broth with yeast and modified tryptic soy broth are suitable base components known in the art. Peptones are various water-soluble protein derivatives obtained by partial hydrolysis of a protein(s) by an acid or enzyme during digestion. Tryptic soy broth generally comprises tryptone (a pancreatic digest of casein), Soytone (a papaic digest of soybean meal) and sodium chloride, for example. Modified tryptic soy broth may further comprise dextrose, bile salts and dipotassium phosphate. Particularly the base broth is selected from the group consisting of tryptone, nutrient broth, L-broth, gram negative broth, peptone, tryptic soy broth, tryptic soy broth with yeast and modified tryptic soy broth. More particularly the base broth is selected from the group consisting of peptone, tryptic soy broth, tryptic soy broth with yeast and modified tryptic soy broth.

In particular embodiments the growth inhibitor is brilliant green, a triarylmethane dye, (CAS number 633-03-4).

Brilliant green is a dye known to inhibit Gram-positive bacteria and a majority of Gram-negative bacilli. It is used in varying amounts in the art, for example 25 mg/L in Difco™ m Brilliant Green Broth, 70 mg/L in Brilliant Green Tetrathionate bile broth, 4.5-6 mg/L in MLCB agar and 10 mg/L in Muller Kauffmann tetrathionate broth. Despite being used for several decades, the inventors have now surprisingly discovered that such concentrations of brilliant green are not optimal for the growth of, for example Salmonella and Shigella. In fact such high levels are believed to be detrimental to the efficient and rapid growth of Salmonella and Shigella and may also impede the recovery of ‘sick’ or ‘stressed’ bacteria. Particular strains of Salmonella such as Salmonella typhi, Salmonella paratyphi amongst others are known as brilliant green sensitive strains and there are currently no suitable culture mediums which do not show a differential inhibitory effect between strains (Chau and Leung, 2008).

The inventors have now discovered a range of concentrations of brilliant green that provide both an inhibitory effect against, for example, gram-positive bacteria whilst allowing the rapid recovery and growth of Salmonella (including S. typhi and S. paratyphi) and Shigella. Thus, in particular embodiments the culture medium comprises brilliant green in an amount of between about 0.05 to about 0.25 mg/L or between about 0.1 mg/L to about 0.25 mg/L, more particularly 0.15 mg/L.

These ‘low levels’ are surprising in light of the levels seen in media already known in the art. It is believed that, due to the long, protracted culture methods known in the art it has previously been necessary to utilise high levels of brilliant green to inhibit the growth of competing microorganisms for the duration of culture which may be as long as 48 hours. However, such is the efficiency of growth in the media of the present invention that microorganisms can be cultured to suitable levels for detection in a single culture medium within 20 hours, particularly about 4-15 hours, more particularly about 4-8 hours and yet more particularly about 4-6 hours. In other embodiments, and for example when used in surface swab testing this may be reduced further from between about 30 minutes to about 4 hours, particularly about 1, 1.5, 2, 2.5 or 3 hours. As a consequence it has been possible to utilise brilliant green at surprisingly low levels which still function to inhibit the growth of certain competing microorganisms for up to 20 hours but which are sufficiently low as to have no effect on growth of the microorganism of interest, such as Salmonella and/or Shigella for example.

Whilst amounts are generally referred to in mg/L or g/L it should be understood that the compositions may be provided pre-mixed in dry form, for example, as tablets, powders, granules or any other convenient dry form to be added to water separately or sequentially. The compositions may also be provided as separate components of a multi package system, if desired. In this case the amounts should be taken to refer to the final concentration of a component that would result once diluted with an appropriate volume of water. For example, a packet of dry powder containing 0.5 mg of brilliant green for dilution in 2 litres of water would have a resultant concentration of 0.25 mg/L.

In other embodiments the medium contains nalidixic acid and/or lithium chloride as growth inhibitor(s).

Nalidixic acid (CAS number 389-08-2) is effective against both gram-positive and gram-negative bacteria. In lower concentrations, it acts in a bacteriostatic manner; that is, it inhibits growth and reproduction of bacteria. In higher concentrations, it is bactericidal, meaning that it kills bacteria instead of merely inhibiting their growth. In particular embodiments the medium contains nalidixic acid in an amount of between about 1 to 3 mg/L, more particularly about 2 mg/L.

Lithium chloride (CAS number 7447-41-8) inhibits the growth of gram-negative bacteria without affecting the growth of gram-positive bacteria. In particular embodiments the medium contains lithium chloride in an amount of between about 1 to 3 g/L, more particularly about 2 g/L.

The use of nalidixic acid and/or lithium chloride as growth inhibitors is beneficial in culture media for the growth of Listeria spp.

In particular embodiments, the culture medium may optionally comprise a growth promoter.

For Salmonella spp, when the base broth consists of peptone, it has been discovered that the inclusion of sodium tetrathionate, or salt thereof, is beneficial. Surprisingly, the Inventors have discovered that levels of sodium tetrathionate in a culture media of above about 20 g/L significantly inhibit the growth of Salmonella spp. This is surprising because levels above 20 g/L are routinely used in the art for positive selection and growth of Salmonella spp. Thus, preferably sodium tetrathionate is present in an amount of between about 1 to about 20 g/L, more particularly about 4 to about 15 g/L, about 6 to about 15 g/L, yet more particularly about 7 to 15 g/L, about 8 to 12 g/L or about 8 g/L.

In alternative embodiments, in place of sodium tetrathionate suitable quantities of sodium thiosulphate and iodine may be used without departing from the spirit of the invention. This is because Iodine may react with sodium thiosulphate to produce sodium tetrathionate (and sodium iodide) in situ. In other embodiments potassium tetrathionate, barium dithionate dehydrate, salts thereof or compounds or mixtures of compounds that release the tetrathionate anion (S₄O₆²⁻) may be utilised.

In other embodiments the culture medium comprises a growth promoter, wherein the growth promoter is ammonium ferric citrate.

In particular embodiments, ammonium ferric citrate (CAS number 1185-57-5) is used in an amount of between about 200 to 1000 mg/L, more particularly about 200 to about 500 mg/L, yet more particularly 200 mg/L to about 300 mg/L and still yet more particularly about 250 mg/L.

In yet another embodiment, the culture medium further comprises the growth promoter sodium citrate.

In particular embodiments, tri-sodium citrate (CAS number 68-04-2) is used in an amount of between about 10 to about 20 g/L, about 12 to 18 g/L and more particularly about 15 g/L.

In particular embodiments, the culture medium is for the growth of Salmonella spp. In other embodiments, the culture medium is for the growth of Shigella spp. In yet further embodiments the culture medium is for the growth of Listeria spp.

According to a second aspect the invention provides a method of releasing the core oligosaccharide monomer from a cell of a microorganism comprising:

(i) adding a detergent to at least one culture sample containing said microorganism to provide a detergent-culture solution; and

(ii) heating the detergent-culture solution to a temperature sufficient to release the core oligosaccharide.

Bacterial lipopolysaccharides (LPS) are an essential component of all gram-negative and some gram-positive bacterial outer membranes. They are believed to be the principle agents responsible for inflammatory responses in patients infected with such bacteria. Examples of gram-negative bacteria include Escherichia coli, Salmonella, Shigella and Campylobacter. Listeria is a gram-positive bacterium.

Most of the characterised LPSs have the same principal structure; the structure of the LPS has been determined as consisting of three distinct regions: a lipid A region, a core oligosaccharide and an o-polysaccharide chain (FIG. 12a). This structure is especially conserved in the lipid A and inner core parts of the LPS. Because of this structural conservation, binding members, such as antibodies, to the lipid A region may not be specific to a particular species leading to false positives in any molecular detection steps. Further, the use of multiple binding members to, for example, the core region is unsatisfactory since such binding members may compete for the same epitope or, because of the close proximity of epitopes, may hinder each other's respective binding reaction. Thus, detection methods of the prior art have relied on binding members specific to the cell surface or flagellae of, for example, Salmonella, since these are easily accessible.

LPSs are generally isolated from bacteria by aqueous phenol extraction followed by purification. Isolated LPSs can then be characterised by, for example, SDS-PAGE, mass spectrometry and NMR (Raetz, 1996). The inventors have discovered that the core oligosaccharide region may be released or made accessible or available for detection, for example by antibody binding techniques, through use of a rapid method utilising a detergent and the application of heat. Use of such a simple methodology would not be suitable for detection of, for example, cell surface antigens or flagellae because detergents are known to interact with lipids and would destroy or disrupt lipid A epitopes with which binding members may react. Whilst detergent alone could be used, the use of heat is further advantageous since it breaks down the LPS into detectable monomers and has the added advantage of killing pathogenic bacteria.

Preferably the detergent is sodium dodecyl sulphate (SDS) or TWEEN 20, 40, 60 or 80.

Surprisingly the inventors have discovered that the use of SDS can enhance binding between a binding member, such as an antibody, and an epitope by as much as 10 fold in the direct assay described below. Similarly, whereas other detergents interfere with and prevent antibody binding in a direct assay (described below), surprisingly the inventors have discovered that TWEEN 20, 40, 60 or 80 has little or no such effect, for example, in a competitive assay. This is in direct contrast to the established teachings of the art, such as in Qualtiere et al, 1977.

The detergent may be added to a culture sample as a liquid, for example, dissolved in a solvent such as water, or in the case of SDS as a solid. Particular detergent concentrations for use in the method are from about 0.1% to about 2%, particularly about 0.5% to about 1% (w/v or v/v).

Preferably the detergent is dissolved or diluted in water and added as a liquid resulting in concentrations described above. Preferably the detergent solution is absent further constituents such as buffers and the like. Thus, in a preferred embodiment, the detergent solution consists essentially of the detergent, either sodium dodecyl sulphate or TWEEN 20, 40, 60 or 80, dissolved in water.

In a next step of the method the detergent-culture solution is heated to a temperature sufficient to release the core oligosaccharide. Preferably the solution(s) is/are heated to a temperature sufficient to kill bacteria, particularly Salmonella, Shigella or Listeria, that may be present in the sample. Particular temperatures include from about 60° C. to about 100° C., particularly about 65, 70, 75, 80, 85, 90, 95 to about 100° C. It will also be apparent to one skilled in the art that steps (i) and (ii) may be carried out sequentially, at the same time, or. that the culture sample and/or detergent may be heated independently before being combined. The detergent-culture solution may be heated for about 30 seconds to about 20 minutes, particularly for about 2 minutes to about 15 minutes, and more particularly for about 2, 3, 4, 5, 6, 7, 8, 9 or about 10 minutes.

In a third aspect of the invention there is provided an assay method for detecting the presence or absence of a microorganism of interest in a test sample, the method comprising:

(i) Culturing the test sample in a culture medium which allows for propagation of the microorganism of interest;

(ii) Treating the test sample sufficient to release one or more core oligosaccharides from any microorganisms present within the test sample;

(iii) Exposing the test sample to at least one binding member which has binding specificity to a core oligosaccharide of the microorganism of interest; and

(iv) Detecting any binding of the at least one binding member to a core oligosaccharide of the microorganism of interest.

The assay method may be direct or indirect. In a direct binding or non-competitive assay (direct or indirect), also referred to as a ‘sandwich assay’, core oligosaccharides are preferably bound to a surface and a binding member, such as an antibody, is reacted with any core oligosaccharides of the microorganism of interest. Preferably the binding member is a labelled binding member. The amount of labelled binding member on the surface is then measured. The results of the direct assay method are generally directly proportional to the concentration of core oligosaccharide in the sample. Clearly the labelled binding member will not bind if the core oligosaccharide is not present in the sample.

In a competitive assay, the core oligosaccharide in the test sample competes with labelled core oligosaccharide for binding to a binding member. The amount of labelled binding member bound to the core oligosaccharide is then measured. In this method, the response will be inversely proportional to the concentration of core oligosaccharide in the sample. This is because the greater the response, the less core oligosaccharide in the ‘unknown’ or test sample was available to compete with the labelled core oligosaccharide.

Regardless of whether the assay is direct or indirect preferably either core oligosaccharide or labelled core oligosaccharide respectively is bound to a surface for detection.

The surface to which the core oligosaccharide(s) are bound may be of a material known in the art, for example, organic polymers such as plastics, glasses, ceramics and the like. Particular organic polymers include polystyrene, polycarbonate, polypropylene, polyethylene, cellulose and nitrocellulose. A preferred polymer is polystyrene and more particularly gamma-irradiated polystyrene. The surface itself may be in the form, or part, of a sheet, microplate or microtitre plate, tray, membrane, well, pellet, rod, stick, tube, bead or the like.

In a particular embodiment LPSs or monomers comprising the core oligosaccharide are immobilised onto a surface without any modification. For example, the hydrophobic lipid A portion of the molecule may bind to a surface, such as a gamma-irradiated polystyrene surface, via non-covalent hydrophobic interactions. Such binding leaves the core oligosaccharide region accessible for interactions with binding members such as antibodies.

In alternative embodiments, the LPSs and/or core oligosaccharides are immobilised onto a surface through use of an intermediate binding member, such as an antibody, conjugate or other linkage. Suitable alternatives are disclosed in International patent application publication no. WO03/36419.

A first step of the method comprises culturing a test sample in a culture medium which allows for propagation of the microorganism of interest.

In certain embodiments, the method is used to detect microbial proteins or fragments present in food or a food product. In further embodiments, the sample is an environmental sample, an agricultural sample, a medical product, or a manufacturing sample. The test sample may be a food product such as meat, meat products including mince, eggs, cheese, milk, vegetables, chocolate, peanut butter and the like including processed, dried, frozen or chilled food products. Alternatively the test sample may be a clinical sample such as a biopsy sample, faecal, saliva, hydration fluid, nutrient fluid, blood, blood product, tissue extract, vaccine, anaesthetic, pharmacologically active agent, imaging agent or urine sample and the like. The test sample may also include swabs, such as skin-, coecum-, faecal, cloacal or rectal-swabs or swabs of surfaces, such as floors, doors and walls or swabs taken from food products including animal carcass swabs. The test sample may also include cosmetic samples such as foundation makeup, lip-balms, lotions, creams, shampoos and the like.

Preferably the test sample is cultured in a culture medium according to the first aspect of the invention.

In particular embodiments the test sample is cultured in a culture medium at about 30° C. to about 44° C., particularly about 37° C. to 42° C., more particularly at about 37° C. The test sample may be cultured in a culture medium for about 4-15 hours, more particularly about 4-8 hours and yet more particularly about 4-6 hours. In other embodiments, the test sample may be cultured in a culture medium from between about 30 minutes to about 4 hours, particularly about 1, 1.5, 2, 2.5 or 3 hours.

A second step of the method comprises treating the test sample sufficient to release one or more core oligosaccharides from any microorganisms present within the test sample.

The test sample may be treated in any way suitable to cause release of bacterial LPSs and or core oligosaccharide from the cell membrane of a microorganism. Preferably the test sample is treated according to the second aspect of the invention.

Other suitable, although possibly less efficient, extraction methods exist in the art and could also be employed including sonication, use of a French press, use of enzymes, ‘bead beating’ and the like. However, the use of detergent with high temperatures (such as boiling or those discussed above) is particularly useful when handling pathogenic bacteria such as Salmonella because high temperatures ensure that all of the bacteria have been killed. More particularly, when the assay is a direct binding assay SDS is preferably utilised whereas when the assay is in the competitive form, SDS is used to prepare the plate coating antigen whilst either TWEEN 20, TWEEN 40, TWEEN 60 or TWEEN 80, particularly TWEEN 20, is employed throughout the rest of the procedure. Suitable heating/treatment time spans are provided in relation to the first aspect above. It will be apparent that the microorganism of interest may not be present in the test sample in which case LPSs and core oligosaccharides of the microorganism of interest will also not be present.

In a third step of the method, the test sample is exposed to at least one binding member which has binding specificity to a core oligosaccharide of the microorganism of interest.

In particular embodiments the core oligosaccharides, LPSs or monomers within the treated test sample are immobilised to a surface prior to step (iii), being exposed to the at least one binding member which has binding specificity to a core oligosaccharide of the microorganism of interest. In such embodiments the core oligosaccharides, LPSs or monomers within the sample may be immobilised by bringing the treated test sample into contact with the surface and incubating and/or maintaining contact for about 10 minutes, 20 minutes, 30 minutes, 40 minutes, 50 minutes to about 60 minutes.

In other embodiments, for example a competitive assay, the test sample is applied to or contacted by a surface on which is already immobilised a known or standard quantity of core oligosaccharide, LPS or monomer. Core oligosaccharide, LPS or monomer from both the known or standard compete with core oligosaccharide, LPS or monomer from the test sample for binding to the at least one binding member.

Core oligosaccharides, LPSs or monomers may be directly immobilised to said surface, for example, by way of non-covalent hydrophobic interactions or indirectly as described above.

The test sample should be exposed to the at least one binding member for a sufficient time to allow for the core oligosaccharide, LPS or monomer to bind to the at least one binding member to form a complex, for example a core oligosaccharide/binding member complex. Suitable times include from about 1 minute to about 4 hours, particularly from about 30 minutes to about 2 hours, particularly about 45 minutes, 1 hour and 1.5 hours.

In certain embodiments, and in an optional step of the method, the complex is exposed to a secondary binding member which has binding specificity to the at least one binding member for a sufficient time to allow for the secondary binding member to form a secondary complex, for example a core oligosaccharide/binding member/secondary binding member complex.

Preferably the binding member is an antibody, more particularly an affinity-purified antibody and yet more particularly a monoclonal antibody.

An antibody for use in the assay of the present invention may be a polyclonal, monoclonal, bispecific, humanised or chimeric antibody. Such antibodies may consist of a single chain but would preferably consist of at least a light chain or a heavy chain, but it will be appreciated that at least one complementarity determining region (CDR) is required in order to bind a target such as a core oligosaccharide or microbial contaminant to which the antibody has binding specificity.

Methods of making antibodies are known in the art. For example, if polyclonal antibodies are desired, then a selected mammal, such as a mouse, rabbit, goat or horse may be immunised with the antigen of choice, such as bacterial endotoxin. The serum from the immunised animal is then collected and treated to obtain the antibody, for instance by immunoaffinity chromatography.

Monoclonal antibodies may be produced by methods known in the art, and are generally preferred. The general methodology for making monoclonal antibodies using hybridoma technology is well known (see, for example, Kohler, G. and Milstein, C, Nature 256: 495-497 (1975); Kozbor et al, Immunology Today 4: 72 (1983); Cole et al, 77-96 in Monoclonal Antibodies and Cancer Therapy, Alan R. Liss, Inc. (1985).

An antibody, as referred to herein, should consist of an epitope-binding region, such as CDR. The antibody may of any suitable class, including IgE, IgM, IgD, IgA and, in particular, IgG. The various subclasses of these antibodies are also envisaged. As used herein, the term “antibody binding fragments” refers in particular to fragments of an antibody or polypeptides derived from an antibody which retain the binding specificity of the antibody. Such fragments include, but are not limited to antibody fragments, such as Fab, Fab′, F(ab′)2 and Fv, all of which are capable of binding to an epitope.

The term “antibody” also extends to any of the various natural and artificial antibodies and antibody-derived proteins which are available, and their derivatives, e.g. including without limitation polyclonal antibodies, monoclonal antibodies, chimeric antibodies, humanized antibodies, human antibodies, single-domain antibodies, whole antibodies, antibody fragments such as F(ab′)2 and F(ab) fragments, Fv fragments (non-covalent heterodimers), single-chain antibodies such as single chain Fv molecules (scFv), minibodies, oligobodies, dimeric or trimeric antibody fragments or constructs, etc. The term “antibody” does not imply any particular origin, and includes antibodies obtained through non-conventional processes, such as phage display. Antibodies of the invention can be of any isotype (e.g. IgA, IgG, IgM i.e. an α, γ or μ heavy chain) and may have a κ (kappa) or a λ (lambda) light chain.

The invention therefore extends to the use of antibodies and antibody derived binding fragments which have binding specificity to core oligosaccharides for use in the present invention.

The term “specifically binds” or “binding specificity” refers to the ability of an antibody or fragment thereof to bind to a target microbial pathogen with a greater affinity than it binds to a non-target epitope. For example, the binding of an antibody to a target epitope may result in a binding affinity which is at least 10, 50, 100, 250, 500, or 1000 times greater than the binding affinity for a non-target epitope. In certain embodiments, binding affinity is determined by an affinity ELISA assay. In alternative embodiments, affinity is determined by a BIAcore assay. Alternatively, binding affinity may be determined by a kinetic method.

In certain embodiments, the binding member, such as an antibody, may be immobilised on the surface and after an optional washing step, the test sample, which may contain the core oligosaccharide or microbial contaminant of interest can be exposed to the surface-bound antibody for a sufficient time for binding to take place and a surface bound first binding member-core complex to form. The assay may then involve a step of exposing the surface bound first binding member-core complex to a secondary binding member, such as an antibody, which may be covalently conjugated with means for light emission, for example, an acridinium ester. In such cases, the secondary binding member has binding specificity for an epitope present on the first binding member, or on the core oligosaccharide or microbial contaminant, so that the amount of signal generated corresponds to the amount of core oligosaccharide or microbial contaminant bound by the primary or secondary binding member.

Typically, an antibody is purified to prevent aggregation.

In certain embodiments the surface is, for example, a microtitre plate of conventional design, but an advantage can be gained by using a modified surface, for instance having darkened side walls and a white or transparent portion (e.g. on the base). This can intensify any signal generated and reduces the background light at the time of measurement. The white portion allows reflection of the light to intensify the generated signal. Thus, in particular embodiments the surface is a multi-well plate comprising a plurality of wells, wherein the base of each well is transparent or substantially transparent, while the walls of the wells are opaque, or darkened to prevent the passage of light, or coloured to provide a contrast against the base portion of the well which allows light to pass there through.

Yet more particularly the antibody is a species specific monoclonal antibody.

Use of the term ‘species specific’ is intended to mean that such an antibody will differentiate between, for example, Salmonella, Shigella and Listeria with little or no cross-reaction.

In particular embodiments, the binding member will interact with and bind to the:

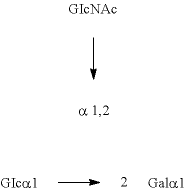

epitope of the LPS core oligosaccharide. This epitope is species specific differentiating Salmonella from other bacteria such as, by way of non-limiting example, Shigella, Listeria, E. coli. In particular embodiments the assay method is a method for the quantitative detection of Salmonella. The assay method may also be utilised to detect for the presence or absence of Salmonella. In particular embodiments the binding member is a labelled binding member labelled by, for example, conjugation to a chemiluminescent or fluorescent compound.

It will be apparent however, that the methods of the invention can be used for identification and quantitation of various target microbial contaminants. The assay methods of the invention involve analysis of samples for the presence or amount of a microbial contaminant. It will be understood that not all samples tested using the methods of the invention will contain microbial contaminants. In certain embodiments, the microbial contaminant is a protein or protein fragment derived from a pathogenic organism. In certain further embodiments, the microbial contaminant may be at least on of the group consisting of, but limited to: a cell wall fragment, a peptidoglycan, a glycoprotein, a lipoprotein, a glycolipoprotein, a small peptide, a sugar sequence and a lipid sequence. The methods of the present invention are particularly suited for detection of microbial proteins including structural proteins and/or toxins derived from bacteria, viruses and fungi.

A fourth step of the method comprises detecting any binding of the at least one binding member to a core oligosaccharide or microbial contaminant of the microorganism of interest.

The detection method may be by any suitable method known in the art such as by fluorescence measurement, colourimetry, flow cytometry, chemiluminescence and the like. In preferred embodiments, detection of binding is by measurement/detection of a luminescent signal, for example, chemiluminescent light produced by a chemiluminescent compound. Suitable chemiluminescent compounds include acridinium esters, acridinium sulfonamides, phenanthridiniums, 1,2-dioxetanes, luminol or enzymes that catalyse chemiluminescent substrates and the like.

In certain embodiments the binding member may be conjugated directly to a light-emitting moiety. In certain embodiments the binding member is conjugated to an acridinium compound or derivative thereof, such as an acridinium ester molecule or acridinium sulphonamide which acts as a luminescent label. In embodiments where the antibody or binding fragment is conjoined to an acridinium ester or acridinium sulphonamide the assay method may further comprise the step of adding AMPPD to the test sample.

AMPPD may also be know by the synonyms: 3-(2′-spiroadamantane)-4-methoxy-4-(3″-phosphoryloxy)phenyl-1,2-dioxetane; 3-(4-methoxyspiro(1,2-dioxetane-3,2′-tricyclo(3.3.1.1(3,7))decan)-4-yl)phenyl phosphate; 4-methoxy-4-(3-phosphatephenyl)spiro(1,2-dioxetane)-3,2′-adamantane.

In certain further embodiments, the antibody may be indirectly associated with a light-emitting moiety, for example the acridinium ester molecule may be conjugated to a second antibody which is capable of binding to the first antibody. In certain embodiments, one or more luminescent or fluorescent moieties may be bound to avidin/streptavidin, which in turn may be bound to biotin chemically conjugated to an antibody. In certain further embodiments, lectins (Protein A/G/L) can be linked to a luminescent or fluorescent molecule which may also be attached to an antibody or other protein conjugate.

The stimulus to produce a detectable signal can be light, for example, of a particular wavelength, e.g. UV light, or may be some other stimulus such as an electrical or radioactive stimulus, a chemical or enzyme-substrate reaction.

Preferably the detection method should be capable of detecting/differentiating 1 colony forming unit (cfu) of Salmonella, Shigella or Listeria in as many as 10,000 cfu of another microorganism such as E. coli, for example, or per swab, starting sample, and the like. Particular detection limits are about 1000 cfu, particularly about 500 cfu, yet more particularly from about 250 cfu, 200 cfu, 150 cfu, 100 cfu, 50 cfu, 10 cfu and about 1 cfu per unit of sample size (mg, g and the like) or volume (ml, L and the like). For liquid cultures a particular detection limit is about 500 cfu/ml.

In other embodiments the antibody may be indirectly associated with such a light-emitting moiety, for example, the acridinium ester molecule may be conjugated to a second binding member which is capable of binding to the first binding member.

The assay methods may be qualitative or quantitative, and standard controls can be run to relate the average signal generated to a given quantity of, for example, core oligosaccharide.

In certain embodiments, the method may be used for the determination in a sample of a plurality of core oligosaccharides or microbial contaminants, this being achieved by providing a plurality of binding members such as antibodies each of which having binding specificity to a different epitope or microbial contaminant. In certain embodiments, antibodies which are bispecific may be used.

It should be apparent that between or at each stage of the method, optional washing, drying and/or incubation steps may be included. The method may also optionally include ‘blocking steps’ between one or more steps of the method wherein a concentrated solution of a non-interacting protein, such as bovine serum albumin (BSA) or casein, is added, for example to all wells of a microtitre plate. Particular blocking agents also include solutions of milk powder and the like. Such proteins block non-specific adsorption of other proteins to the plate and may be beneficial in reducing ‘background’ artifacts which can interfere with the sensitivity of the assay.

According to a fifth aspect of the invention there is provided the use of a binding member which has binding specificity to a core oligosaccharide for the specific detection of a microorganism selected from the group consisting of Salmonella, Shigella and Listeria.

According to a sixth aspect of the invention there is provided a kit for carrying out the invention according to the first, second, third, fourth and/or fifth aspect of the invention. Such kits may comprise culture media in liquid (ready-to-use or concentrated for dilution) or dry (for example, powder, granules, tablets, etc.) form, detergents or detergent solutions, wash buffers, diluents, pre-prepared plates, tubes or beads, one or more antibodies (i.e. primary, secondary), detection reagents, gloves, pipette tips, instruction manuals and the like. Wells of pre-prepared plates or tubes may be pre-coated with a known or standard amount of a core oligosaccharide, LPSs or monomer or a binding member such as an antibody. Such pre-prepared surfaces may be lyophilised.

### BRIEF DESCRIPTION OF FIGURES

FIGS. 1(a) and (b) are schematics of a direct binding assay wherein ♦ represents the bacterial core-oligosaccharide, LPSs or monomer, for example, of Salmonella. FIG. 1(a) shows a direct immunoassay, FIG. 1(b) shows an indirect immunoassay.

FIGS. 2(a) and (b) are schematics of a competitive binding assay wherein ♦ represents the bacterial core-oligosaccharide, LPSs or monomer, for example, of Salmonella. FIG. 2(a) shows a direct competitive immunoassay, FIG. 2(b) shows an indirect competitive immunoassay.

FIG. 3 is a graph demonstrating the positive growth effect of tetrathionate on Salmonella whilst growth of other bacteria is inhibited.

FIG. 4 is a graph demonstrating the effect of brilliant green on growth of Salmonella, Shigella, E. coli and staphylococcus. The graph exemplifies the optimum range of concentrations of brilliant green for growth of Salmonella with inhibition of competing bacteria, particularly at levels of 0.15 mg/l brilliant green.

FIG. 5 is a graph demonstrating the effect of ferric ammonium citrate on growth of Salmonella, Shigella, E. coli and staphylococcus. The graph exemplifies the optimum range of concentrations of ferric ammonium citrate for growth of Shigella particularly at levels of 0.25 g/l. At levels above 0.25 g/l, growth of Salmonella is unaffected.

FIG. 6 is a graph demonstrating the effect of sodium citrate on growth of Salmonella, Shigella, E. coli and staphylococcus. Whilst growth of both Salmonella and Shigella is enhanced, growth of competing bacteria is inhibited.

FIG. 7 is a graph demonstrating bacterial growth in Gram-Negative broth.

FIG. 8 is a graph demonstrating bacterial growth in deoxycholate citrate lactose sucrose broth.

FIG. 9 is a graph demonstrating bacterial growth in Peptone Broth.

FIG. 10 is a graph demonstrating bacterial growth in modified Tryptic Soy Broth. The growth of both Salmonella and Shigella is enhanced demonstrating a doubling time of ˜30 minutes.

FIG. 11 is a graph demonstrating high growth of Listeria spp. With inhibition of competing bacteria in broths of the present invention.

FIG. 12(a) illustrates the general structure of the LPS(O-antigen, core polysaccharide (oligosaccharide), lipid A) of certain bacteria of interest. FIG. 12(b) is a detailed illustration of the Salmonella LPS monomer including the species specific antibody binding epitope.

### DETAILED DESCRIPTION OF INVENTION

The assays of the present invention are preferably utilised to identify the presence or absence of core oligosaccharides of bacterial LPSs in a given sample. The assays of the present invention are capable of identifying samples containing, or contaminated, with bacteria such as Salmonella, Shigella or Listeria which have species-specific epitopes in the core oligosaccharide region of the LPS. The inventions may be better appreciated by reference to the following description and examples which are intended to be illustrative of the methods of the invention.

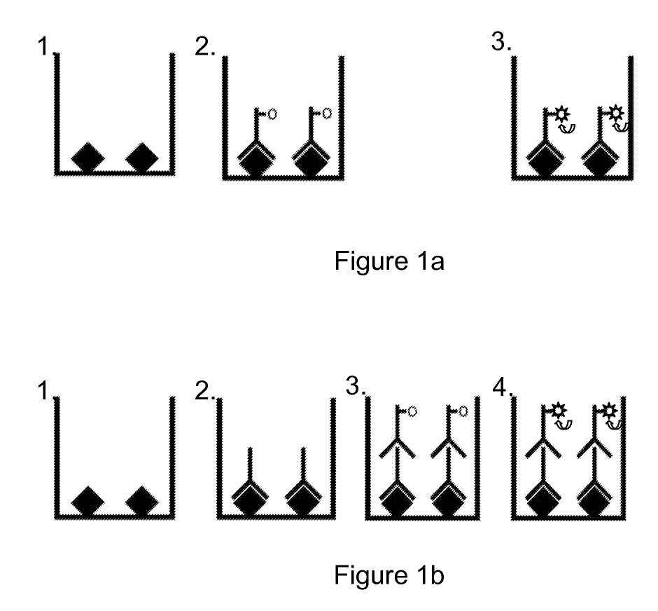

FIG. 1a illustrates the steps of a direct binding assay utilising a labelled primary antibody. FIG. 1b illustrates the direct binding assay utilising unlabelled primary antibody and a secondary labelled antibody. A direct binding (direct or indirect antibody-linked) chemiluminescence-based immunosorbent assay for the detection of Salmonella spp on animal carcasses and in foodstuffs may be carried out as described below.

25 g of a food sample were added to 225 ml culture medium according to the first aspect of the invention. Alternatively a surface swab may be taken from a 10×10 cm area on a carcass and cultured in 2-5 ml culture medium according to the first aspect of the invention. Specifically the culture medium comprised 1% peptone, 8 g/L sodium tetrathionate and 0.15 mg/L brilliant green. The sample was cultured for 5 hours at 37° C.

After 5 hours of culture, a 2 ml aliquot of the sample was removed and SDS was added to a final concentration of 0.5% (w/v). The sample was heated to 100° C. for 5 minutes and allowed to cool. One hundred microlitres of each test sample was added directly to a well of a solid white 96 well high binding microtitre plate (Greiner Bio One) and incubated at 37° C. for 30 minutes. During incubation, the lipid A portion of the LPS binds to the surface of the plate via non-covalent hydrophobic interactions (FIG. 1a (1), FIG. 1b(1)). Following incubation the plate was emptied and the wells washed three times with a wash buffer comprising 0.01M sodium phosphate buffer, pH 7.4, containing 0.147M NaCl and 0.05 (v/v) Tween 20.

One hundred microlitres of anti-Salmonella antibody conjugate, at a concentration of 500 ng/ml in 0.01M phosphate buffer, pH7.4, containing 0.147M NaCl, was added to each well. The final concentration of antibody per well was 50 ng. The plate-bound sample (Salmonella LPS/core oligosaccharide) and antibody were incubated in the coated wells for 30 minutes as 37° C. Following incubation, plates were washed three times in wash buffer and pat dried prior to detection (FIG. 1a(2), FIG. 1b(2)).

When the anti-Salmonella is directly labelled with acridinium ester, plates were placed into a luminometer. 30 μl of trigger solution A and 60 μl of trigger solution B was added to each well of the microtitre plate to initiate light output from conjugated acridinium ester (FIG. 1a(3)). The luminometer settings were as follows:

Delay Injection P (for solution A)—1.6 seconds
Measurement Time Interval 1—0.0 seconds
Delay injection M (for solution B)—0.0 seconds
Measurement Time Interval 2—1.0 seconds

Trigger solution A comprised: 63 μl 70% (w/w) HNO3 and 165 μl 30% (v/v) H2O2 in a total volume of 10 ml distilled water. Trigger solution B comprises: 0.1 g NaOH and 75 mg CTAC in 10 ml of distilled water.

### Addition of Goat Anti-Mouse IgG2b Acridinium Conjugate (if Anti-Salmonella Monoclonal Antibody is Unconjugated).

When the anti-Salmonella antibody is not labelled, a second binding member, a goat anti-mouse IgG2b conjugate is used. Post column IgG2b was diluted 1:100 in a diluent comprising 3% (w/v) non-fat milk powder and 0.05% (v/v) Tween 20 and 100 ul of this solution was added to each well of the plate (FIG. 1b(3)). Following incubation at 37° C. for 60 minutes the plate was washed four times in wash buffer, dried and read as above (FIG. 1b(4)).

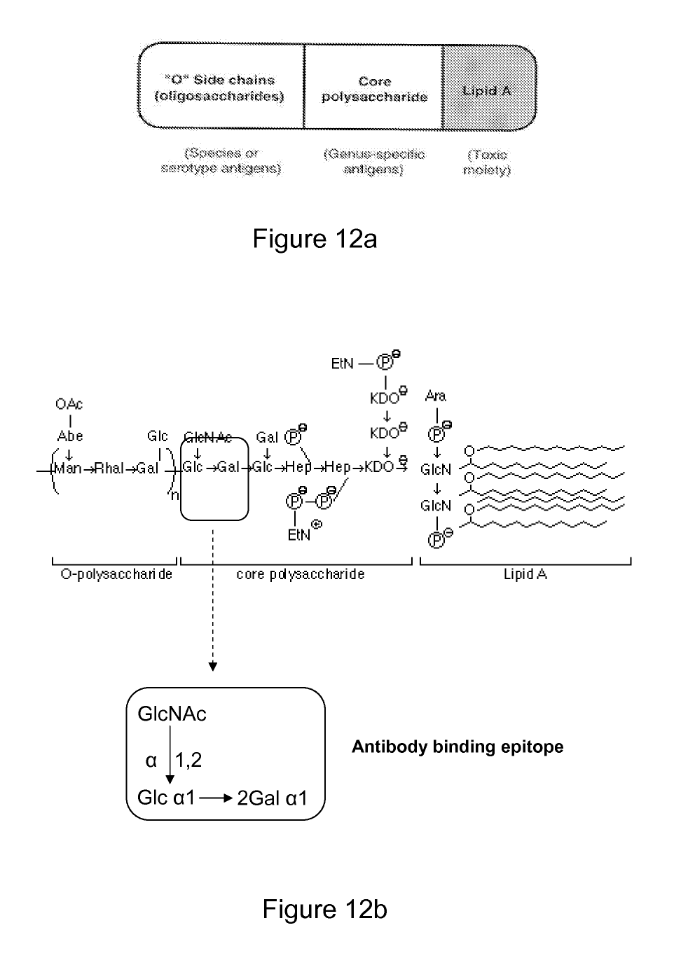

A competitive (direct or indirect) chemiluminescence-linked immunosorbent assay for the detection of Salmonella spp in foodstuffs may be carried out as described below. Salmonella enteritidis LPS-coated microtitre plates were prepared as follows. S. enteritidis was cultured in a standard broth culture medium (2% (w/v) Buffered Peptone Water—Oxoid) not according to the first aspect of the invention for 18 hours. The number of colony forming units was quantified and approximately 10⁸ cfu/ml were placed in a covered but unsealed polypropylene boiling tube containing NaEDTA and SDS to achieve final concentrations of 10 mM and 0.5% (w/v) respectively. The culture was boiled at a temperature of 100° C. for 2 minutes thereby killing the bacteria (and neutralising any biohazard associated) whilst also exposing the bacterial LPS core oligosaccharide or monomer epitope (see for example FIG. 12b). The boiled stock was further diluted to a concentration of 10⁶ cfu/ml by addition of a diluent comprising 2% Buffered Peptone Water (BPW).

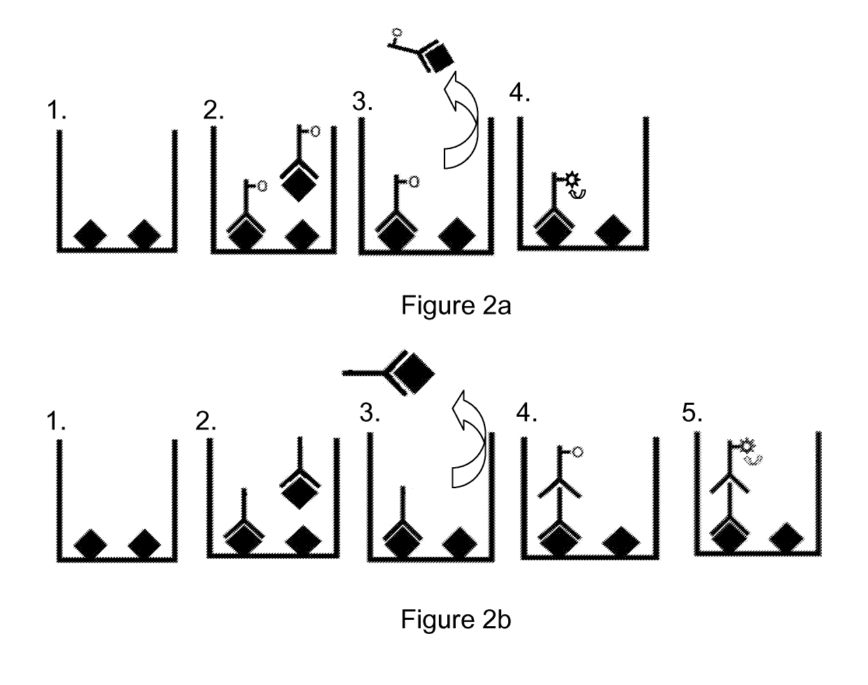

One hundred microlitres of diluted boiled stock was added to each well of a solid white 96 well high binding microtitre plate (Greiner Bio One) and incubated at 37° C. for 60 minutes. During incubation, the lipid A portion of the LPS binds to the surface of the plate via non-covalent hydrophobic interactions. Following incubation the plate was emptied and the wells washed three times with a wash buffer comprising 0.01M sodium phosphate buffer, pH 7.4, containing 0.147M NaCl and 0.05 (v/v) Tween 20. Washed coated plates were either used immediately or freeze-dried for storage (FIG. 2a(1), FIG. 2b(1)).

25 g of a test sample of minced meat spiked with 10 cfu of Salmonella was added to 200 ml of culture medium according to the first aspect of the invention. Specifically the culture medium comprised 1% peptone, 8 g/L sodium tetrathionate and 15 mg/L brilliant green. The sample was cultured for 5 hours at 37° C. After 5 hours of culture, a 5 ml aliquot of the sample was removed and TWEEN 20 was added to a final concentration of 2% (v/v). The sample was heated to 100° C. for 2 minutes and allowed to cool. 80 ul aliquots of the boiled sample were added to each well of the coated microtitre plate.

Twenty microlitres of anti-Salmonella antibody conjugate at a concentration of 125 ng/ml in 0.01M phosphate buffer, pH7.4, containing 0.147M NaCl was added to each well (FIG. 2a(2), FIG. 2b(2)). The final concentration of antibody per well was 25 ng/ml. Competing sample LPS/core oligosaccharide and antibody were incubated in the coated wells for 60 minutes as 37° C. Following incubation, plates were washed three times in wash buffer and pat dried prior to detection (FIG. 2a(3), FIG. 2b(3)).

When the anti-Salmonella is directly labelled with acridinium ester, plates were placed into a luminometer. 30 μl of trigger solution A and 60 ul of trigger solution B was added to each well of the microtitre plate to initiate light output from conjugated acridinium ester (FIG. 2a(4)). The luminometer settings were as follows:
Delay Injection P (for solution A)—1.6 seconds
Measurement Time Interval 1—0.0 seconds
Delay injection M (for solution B)—0.0 seconds
Measurement Time Interval 2—1.0 seconds

Trigger solution A comprised: 63 ul of 70% (w/w) nitric acid (HNO₃) and 165 ul of 30% (v/v) H₂O₂ in a total volume of 10 ml of distilled water. Trigger solution B comprises: 0.1 g NaOH and 75 mg of CTAC in 10 ml of distilled water.

### Addition of Goat Anti-Mouse IgG2b Acridinium Conjugate (if Anti-Salmonella Monoclonal Antibody is Unconjugated)

When the anti-Salmonella is not labelled, a second binding member, a goat anti-mouse IgG2b conjugate is used. Post column IgG2b was diluted 1:100 in a diluent comprising 3% (w/v) non-fat milk powder and 0.05% (v/v) Tween 20 and 100 ul of this solution was added to each well of the plate (FIG. 2b(4)). Following incubation at 37° C. for 60 minutes the plate was washed four times in wash buffer, dried and read as above (FIG. 2b(5)).

### EXAMPLES

### Example 1

### Preparation of Culture Media for Growth of Salmonella

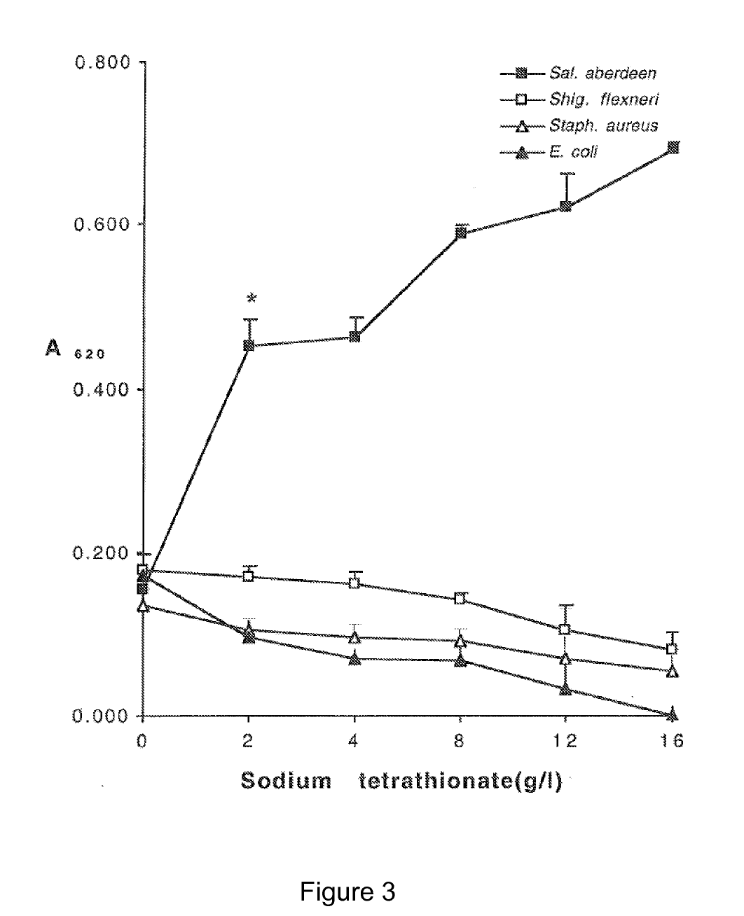

FIG. 3 demonstrates the effect of sodium tetrathionate at concentrations of between 0 and 16 g/L on the growth of Salmonella aberdeen, Shigella flexneri, Staphylococcus aureus and E. coli. 0.1 ml inoculum (10³ cells/ml) was added to a 100 ml conical flask containing tryptic soy broth with 0 to 16 g/L of sodium tetrathionate. The flask was incubated at 37° C. for 18 hours. After this time, the A₆₂₀ was measured. Each value represents the mean±SD of three separate experiments. * shows p<0.05. At levels of between 2 to 16 g/L growth of Shigella, Staphylococcus and E. coli are inhibited in contrast to growth of Salmonella which is un-affected or promoted.

| Concentration | | | | |
|---|---|---|---|---|
| (g/litre) | E. coli | A₆₂₀ | Salmonella | A₆₂₀ |
| 0 | 0.214 | 0.208 | 0.156 | 0.138 |
| 4 | 0.096 | 0.104 | 0.187 | 0.179 |
| 8* | 0.078 | 0.073 | 0.818 | 0.848 |
| 12 | 0.053 | 0.048 | 0.226 | 0.270 |
| 15 | 0.011 | 0.011 | 0.167 | 0.186 |
| 20 | 0.015 | 0.018 | 0.150 | 0.139 |
| 25 | 0.023 | 0.022 | 0.086 | 0.099 |
| 30 | 0.021 | 0.020 | 0.059 | 0.073 |

Not only does the Tetrathionate inhibit the growth of E. coli at levels of >4 g/litre but at a concentration of 8 g/litre it has a clear enhancing effect on the growth of Salmonella. N.B. A₆₂₀ measures turbidity and hence, the higher the value the higher the bacterial growth. At levels above 16 g/L, growth of Salmonella is inhibited.

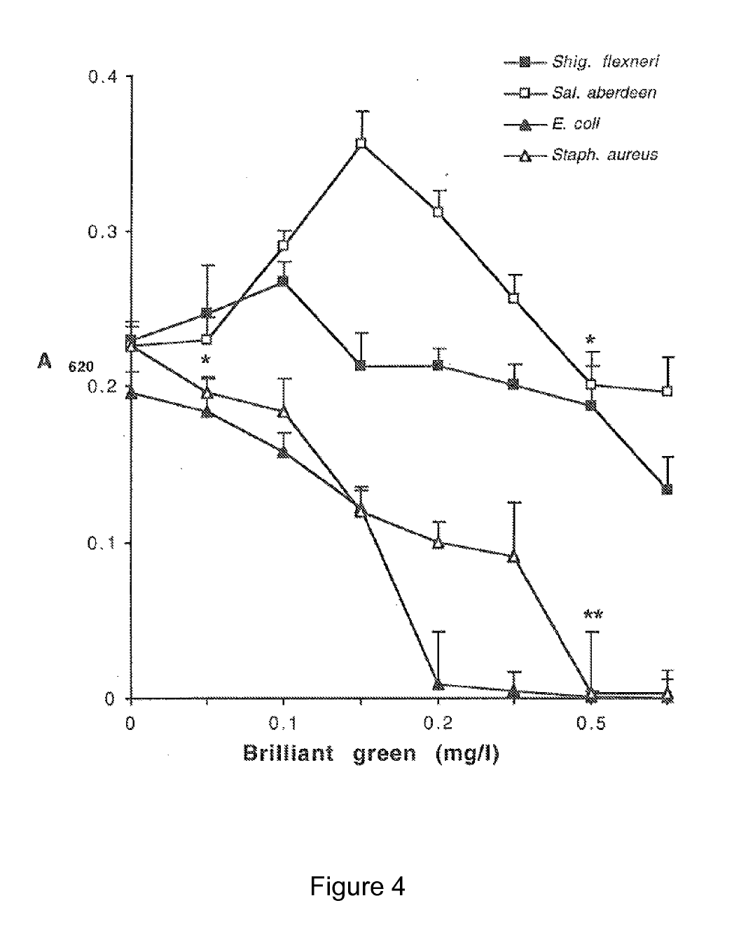

FIG. 4 demonstrates the growth response of bacteria to brilliant green. 0.1 ml inoculum (10³ cells/ml) was added to a 100 ml conical flask containing tryptic soy broth with 0.05 g to 5 g/L of brilliant green. The flask was incubated at 37° C. for 18 hours. After this time, the A₆₂₀ was measured. Each value represents the mean±SD of three separate experiments. * shows p<0.05, **p<0.01.

| Concentration | | | | |
|---|---|---|---|---|
| (mg/litre) | E. coli | A₆₂₀ | Salmonella | A₆₂₀ |
| 0 | 0.235 | 0.279 | 0.256 | 0.238 |
| 0.15 | 0.102 | 0.118 | 0.255 | 0.229 |
| 0.3 | 0.043 | 0.062 | 0.046 | 0.041 |
| 1 | 0.037 | 0.035 | 0.057 | 0.040 |
| 3 | 0.041 | 0.034 | 0.072 | 0.061 |
| *5 | 0.250 | 0.230 | 0.282 | 0.256 |
| *7 | 0.524 | 0.500 | 0.606 | 0.569 |
| *10 | 0.624 | 0.665 | 0.614 | 0.607 |

A₆₂₀ is employed as a measure of bacterial numbers by a turbidometric method. *high absorbance values due to absorbance of Brilliant Green; at these concentrations the spectrometer could not be blanked against the Brilliant Green solution. At levels of Brilliant Green 0.3 mg/L or higher the growth of both Salmonella and E. coli are limited but at 0.15 mg/L it has an inhibitory effect on the E-coli but NOT the Salmonella.

### Example 2

### Preparation of Culture Media for Growth of Shigella

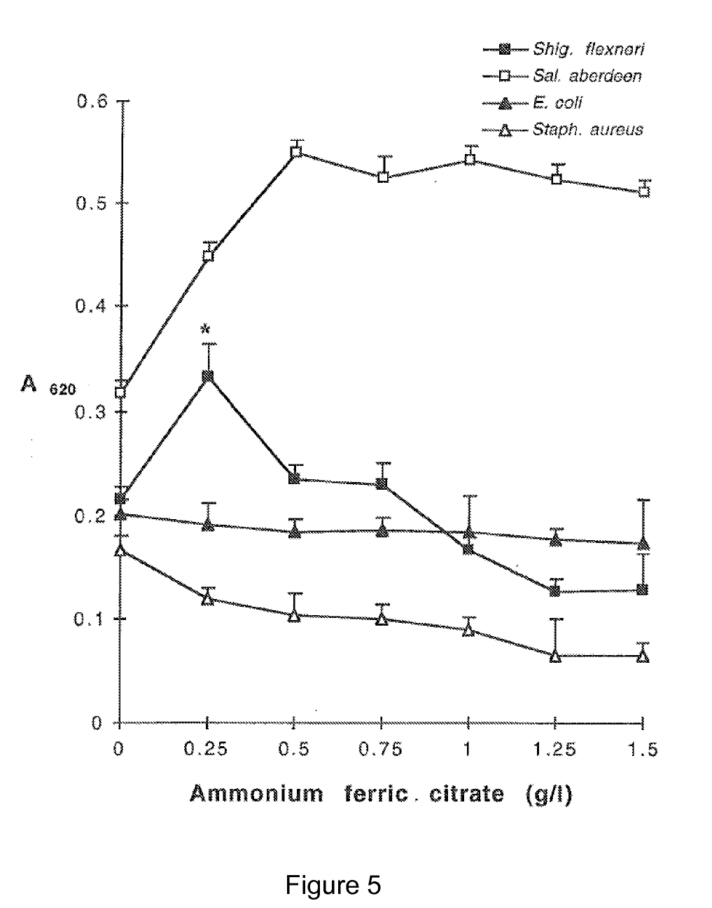

FIG. 5 demonstrates the growth response of bacteria to ammonium ferric citrate. 0.1 ml inoculum (10³ cells/ml) was added to a 100 ml conical flask containing tryptic soy broth with 0.25 to 1.5 g/L of ammonium ferric citrate. The flask was incubated at 37° C. for 18 hours. After this time, the A₆₂₀ was measured. Each value represents the mean±SD of three separate experiments. * shows p<0.05. At levels of ammonium ferric citrate of 0.25 g/L or higher the growth of both Staphylococcus and E. coli are limited.

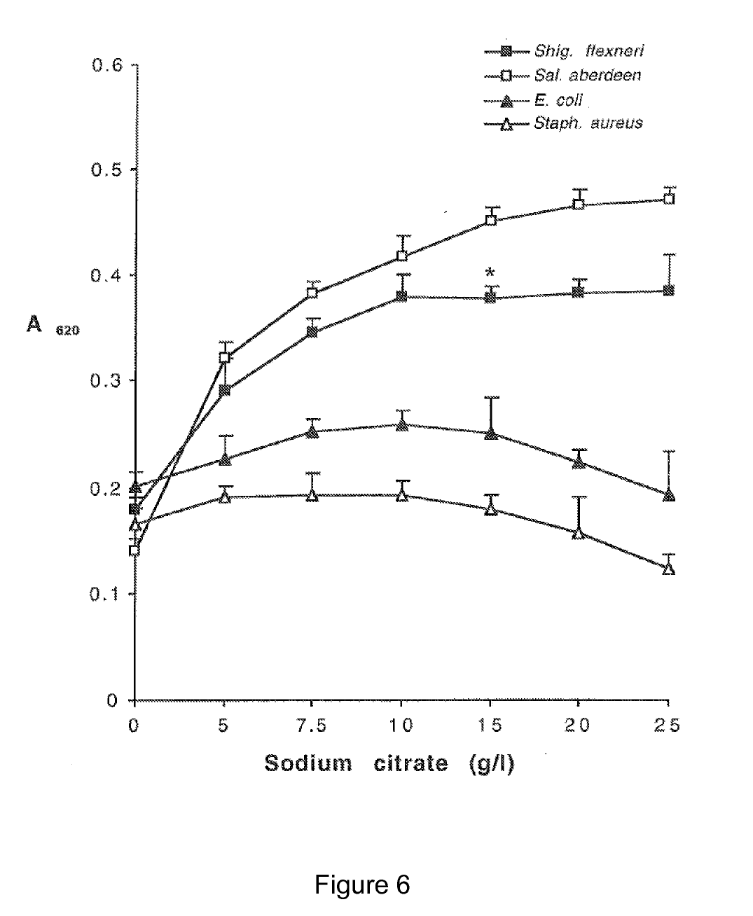

FIG. 6 demonstrates the growth response of bacteria to sodium citrate. 0.1 ml inoculum (10³ cells/ml) was added to a 100 ml conical flask containing tryptic soy broth with 5 to 25 g/L of sodium citrate. The flask was incubated at 37° C. for 18 hours. After this time, the A₆₂₀ was measured. Each value represents the mean±SD of three separate experiments. * shows p<0.05. At levels of sodium citrate of 5 g/L or higher the growth of both Staphylococcus and E. coli are limited. At levels of 15 g/L the growth response of Shigella is significantly increased over those of Staphylococcus and E. coli.

### Example 3

### Generation Study of Different Bacteria in Peptone, Tryptic Soy Broth and Modified Tryptic Soy Broth

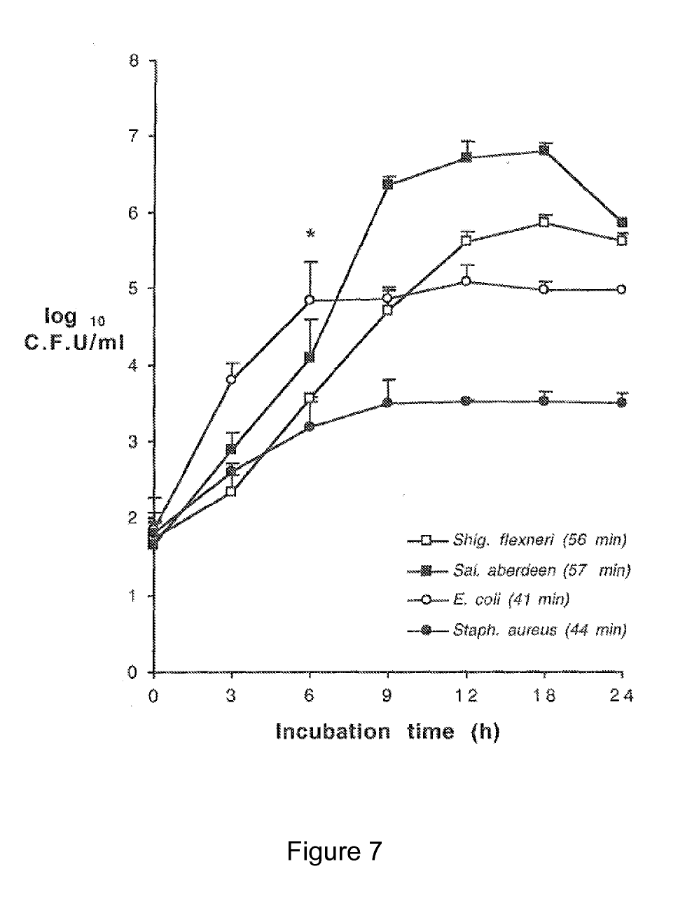

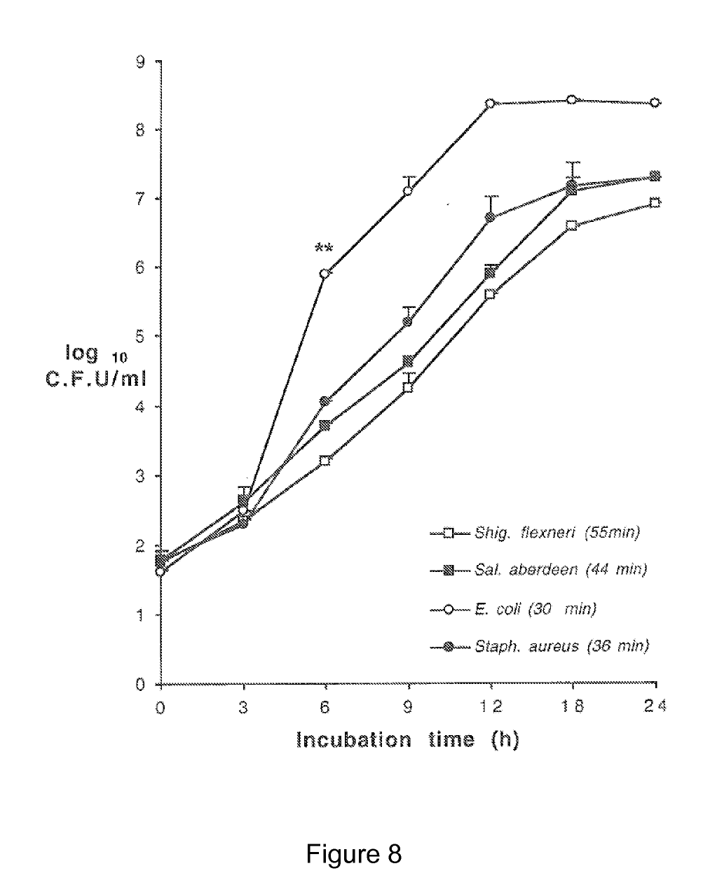

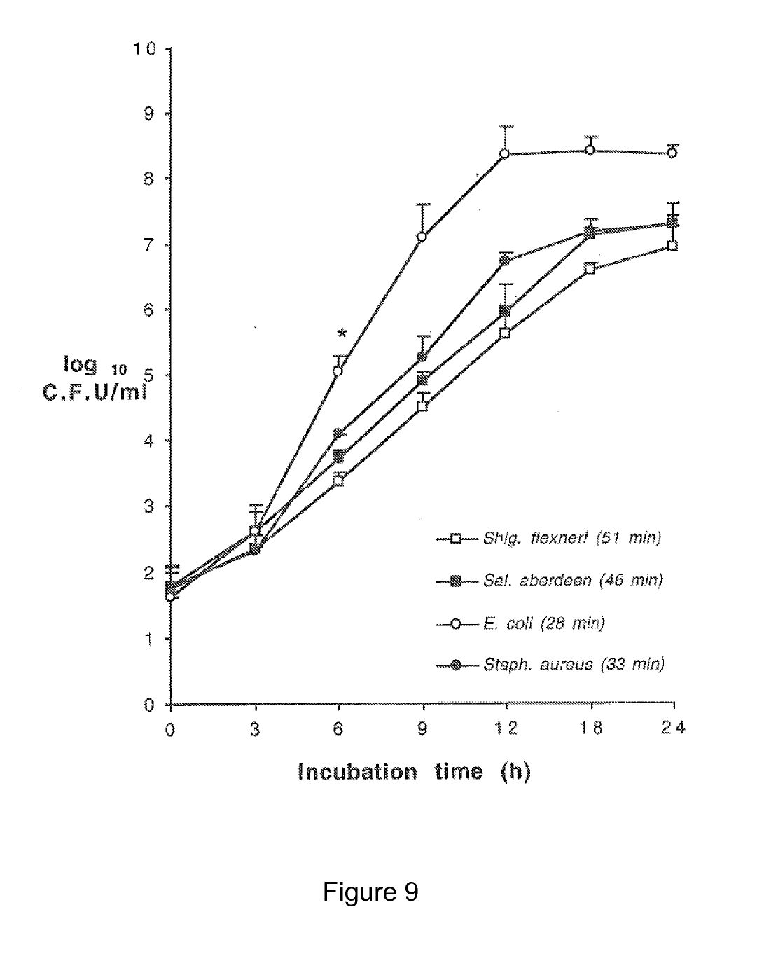

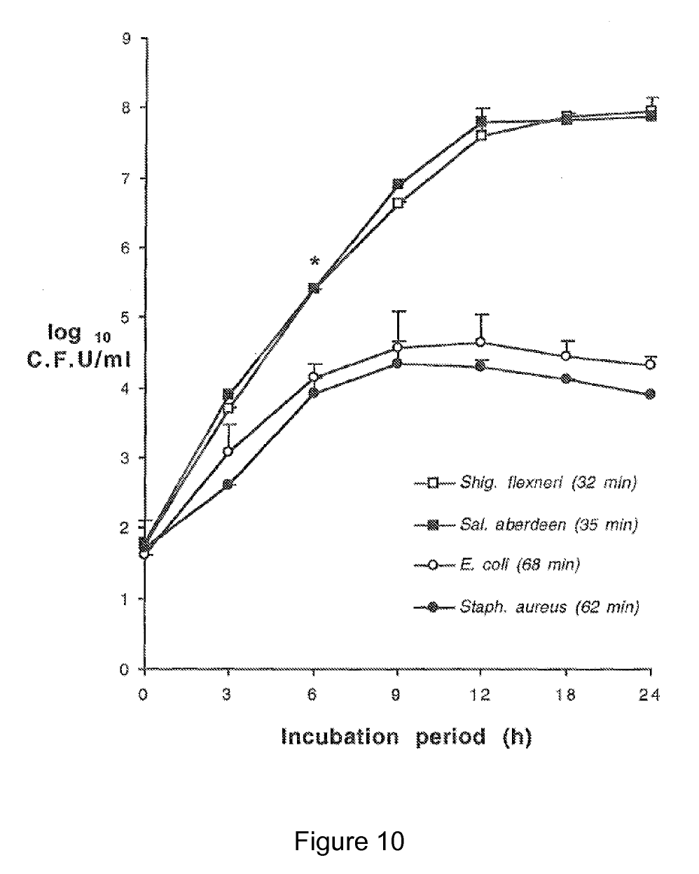

Three strains of Shigella and other bacteria including Salmonella aberdeen, E. coli and Staphylococcus aureus were grown in conventional broth cultures to investigate the generation time. 0.1 ml inoculum (10³ cells/ml) was added to a 100 ml conical flask containing either peptone (FIG. 7), trypric soy broth (TSB) (FIG. 8), modified tryptic soy broth (mTSB) (FIG. 9) or gram-negative broth (FIG. 10).

Each flask was incubated at 37° C. for 18 hours. After this time, the number of viable cells was determined by drop plate technique on nutrient agar. The values in parenthesis are generation times. Each value represents the mean±SD of three separate experiments. * shows p<0.05. The doubling time was studied in peptone, tryptic soy and modified tryptic soy broth. The generation time of Shigella flexneri, Salmonella aberdeen, E. coli and Staphylococcus aureus was 36, 57, 41 and 44 min respectively when they were grown in Gram-negative broth.

The growth rate of all bacteria increased in TSB. As a result, TSB was used as the basic growth media in conjunction with other traditional selective agents, alone or in combination to selectively allow better growth of Shigella. The doubling time of Shigella flexneri, Salmonella aberdeen, E. coli and Staphylococcus aureus was 48, 46, 28 and 33 min in TSB. Shigella flexneri and Salmonella aberdeen grew significantly better (p<0.01) in the mTSB, whereas, it took longer for E. coli and Staphylococcus aureus to multiply in this base broth. The growth rate of E. coli could be delayed up to 68 min when grown in modified TSB. Generation time of Shigella flexneri could be shortened to 46 minutes in modified TSB.

### Example 4

### Preparation of Culture Media for Growth of Listeria

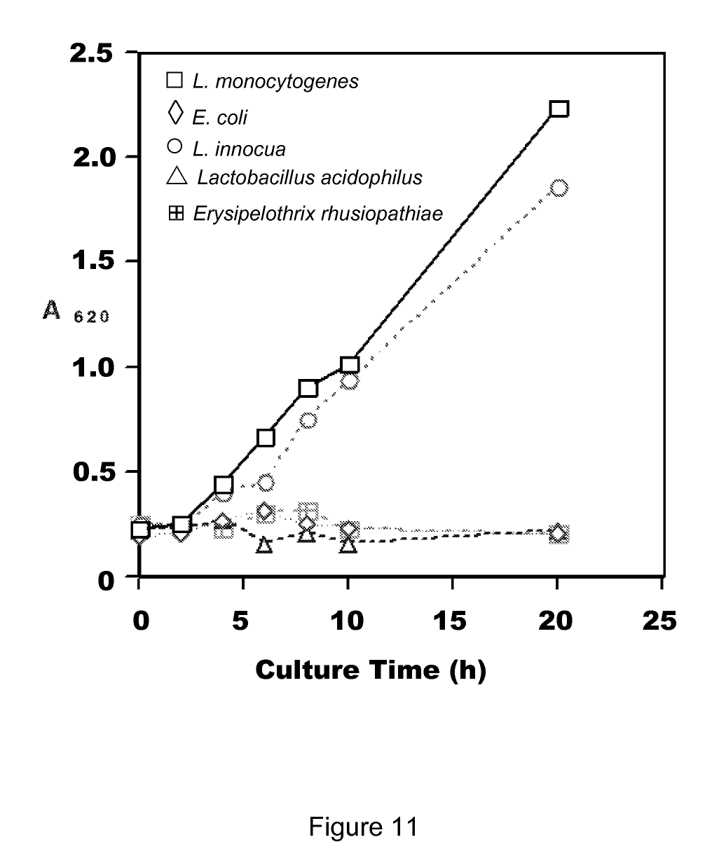

Listeria growth medium was prepared comprising a combination of lithium chloride and Nalidixic acid. 0.1 ml inoculum (10³ cells/ml) was added to a 100 ml conical flask containing according to the following recipe: TSBYE—3.3% tryptic soy broth with 0.5% yeast extract, 2 g/l LiCl, 2 mg/l Nalidixic acid and 250 mg/l ammonium ferric citrate. The flask was incubated at 37° C. for 20 hours. After this time, the A₆₂₀ was measured (FIG. 11). Each value represents the mean±SD of three separate experiments. L. monocytogenes and L. innocua were both able to grow efficiently in the media. The growth of E. coli. Lactobacillus acidophilus and Erysipelothrix rhusiopathiae were all significantly inhibited.

### Example 5

### Preparation of Culture Media for the Selective Growth of Salmonella, Shigella or Listeria

Utilising the above data, selective culture media were prepared according to the following recipes:

### Salmonella 1

2% peptone
0.15 mg/l brilliant green
4-8 g/l tetrathionate (All types)

### Salmonella 2

3.3% (w/v) mTSB
0.15 mg/l BG
1 g/l ammonium ferric citrate.

### Shigella 1

3.3% mTSB
0.1 mg/l Brilliant Green
250 mg/l ammonium ferric citrate
15 g/l trisodium citrate

### Listeria 1

TSBYE—3.3% tryptic soy broth with 0.5% yeast extract

### 2 g/l LiCl

2 mg/l Nalidixic acid
250 mg/l ammonium ferric citrate

### Example 6

### Preparation of Antibodies and Antibody Fragments for Conjugation with Acridium Ester for Use in Immunoassays

An improved preparation of antibodies can be produced by preparation of pure IgG from ascites by Protein A chromatography, followed by the optional step of cleaving the IgG to give a Fab fragment, and conjugation of the fragment or whole antibody to an ester and subsequent purification. Alternatively, other isotypes or isoforms of antibody can be used unpurified.

### Protein A/G Separation

### (i) Buffers and Solutions

PBS: 0.1 M phosphate buffer, pH 8, containing 0.15M NaCl.

0.1M citrate-acid buffers, pH 6, and pH 4.5: dissolve 29 g dry sodium citrate in 800 ml of distilled water. Add 1 M citric acid solution (210 g/l) until a pH of 6 and 4.5, respectively, is obtained. Make up to 1 litre.

pH 3 buffer, 0.1M acetic acid containing 0.15 M NaCl, to 800 ml of distilled water, add 100 ml 1 M acetic acid and 100 ml 1.5 M NaCl.

1.5 M glycine buffer, pH 8.9, containing 3M NaCl: dissolve 112 g glycine and 174 g NaCl in 700 ml of distilled water. Adjust pH to 8.9 with 5M sodium hydroxide solution and make up to 1 litre with distilled water.

### (ii) Procedure

Allow 1.5 g of Protein A-Sepharose (CL-4B, Pharmacia) (Protein G may also be used) to swell in 0.1 M phosphate buffer, pH 8, for 30 minutes (1.5 g of beads give 5 ml of gel); fill a 20×2 cm column and rinse with starting buffer. After dialysis against starting buffer, load on to the column 1 ml delipided ascites previously precipitated by ammonium sulphate at 40% (v/v) saturation. Delipiding of ascites, if used, is carried out by centrifugation at 100,000 g for 45 minutes. Any pellet formed or floating ‘lipid’ is discarded. Wash the column with 0.1 M phosphate buffer, pH 8, until the A₂₈₀ is <0.050. Add citrate buffer, pH6, and wash until the A₂₈₀ is <0.050. Elute the other immunoglobulins in the same manner by successively employing the pH4.5 and pH 3 buffers. Neutralise with phosphate buffer, pH 8, containing 0.02% sodium azide, and store the Protein A-Sepharose in this buffer. After elution, neutralise the antibody with several drops of 1M phosphate buffer, pH 8, and dialyse against PBS.

(iii) Preparation of Fab Fragments

Since enzymatic digestion never goes to completion, the action of papain on IgG gives rise to 10% of undigested IgG in addition to the Fab and Fc fragments, which have the same molecular weight and are hence difficult to separate. Protein A is used to simplify their purification. In the first step, the antibody is treated with papain, and then the mixture is passed over Protein A in order to isolate the IgG and the Fab. The Fab fragments are then separated from undigested IgG by filtration on Sephadex or Protein A-Sepharose.

### Materials

Chromatography column (2.5 cm×80 cm).

### Papain: Boehringer.

L-cysteine hydrochloride: Merck.
EDTA: ethylenediaminetetraacetic acid: Merck.

### Iodoacetamide: Merck

### Buffers and Solutions

Phosphate-buffered saline—PBS.
0.1M phosphate buffer, pH 7.4.
1 mg/ml papain solution prepared from a commercial stock solution.
0.2M L-cysteine: 35 mg/ml of 0.1M phosphate buffer, pH 7.4.
0.1M EDTA: dissolved 3.6 g EDTA in 100 ml of 0.2M NaOH. Since EDTA dissolves significantly only at approximately pH 8, it may be necessary to add a few drops of IM NaOH in order to pH the solution to obtain complete solubility of the EDTA.
0.4M iodoacetamide: 74 mg/ml in 0.1M phosphate buffer, pH 7.4.

### Procedure

The immunoglobulin fraction was prepared from the antiserum by precipitation with 40% (v/v) saturated ammonium sulphate solution. The immunoglobulins were dialysed against 0.1M phosphate buffer, pH 7.4. An approximate determination of the protein concentration was made (a 1 mg/ml solution of IgG gives an A₂₈₀ of 1.4).

The concentration of the IgG was adjusted to 30 mg/ml and the final volume (V) required to give a protein concentration of 20 mg/ml was calculated. For affinity purified antibodies, a final concentration of 2.5 mg/ml was used. A volume of V/20 of 0.04M EDTA (final concentration: 0.002 M) was added. A volume of V/20 of 0.2M L-cysteine solution (final concentration: 0.01M) was then added. A 1 mg/ml papain solution to give 1 mg of papain per 100 mg globulins was added. The volume was adjusted to V ml with the 0.1M phosphate buffer, pH 7.4. The reaction was allowed to proceed for 2 hours at 37° C. A volume of V/10 of a 0.4M iodoacetamide solution (final concentration: 0.04M) was added. This was left for 30 minutes, and then the preparation was dialysed against PBS overnight at +4° C.

IgG antibodies binding to the GlcNAc-Glc-Gal epitope (FIG. 12) were isolated and the Fab fragments were isolated by Protein A chromatography. The mixture was fractionated on a column of Sephadex G-100 (2.5×80 cm) and equilibrated with PBS. The first peak corresponded to IgG, and the second peak corresponded to the Fab fragments. The Fab peak was concentrated to 5 mg/ml.

### Example 7

### Conjugation of the Antibody or Antibody Fragment with Acridinium Ester

### a) Preparation

i) The acridindium ester e.g. (4-(2-succinimidyloxycarbonylethyl)phenyl-10-dimethylacridinium-9-carboxylate fluorosulphate is weighed in a clean, dry borosilicate vial. Dry dimethyl formamide is added (volume depending on acridinium ester quantity available) and the solution aliquoted into vials at 5 mg per vial normally.

ii) Antibody is dissolved in 0.2M sodium phosphate buffer, pH 8.0 at a concentration of 0.5 mg IgG/ml.

iii) Add 5 mg acridinium ester solution to 200 ml antibody solution and mix well.

iv) Incubate for 15 minutes at room temperature and then stop reaction by the addition of 100 ml 10% (w/v) lysine monohydrochloride followed by a further 5 minutes at room temperature in the dark.

v) Purify in accordance with (b) below.

### b) Purification of Conjugate

(i) A gel filtration column may be used to purify the conjugate.

A column of 1.6×100 cm of Sephadex G200 (Pharmacia) is equilibrated with 0.1M phosphate buffer, pH 7.4, containing 0.147 M NaCl and 0.5% (w/v) bovine serum albumin (Sigma). Up to 1.5 ml of conjugate is placed on the column and separated for 18 hours at a flow rate of 9 ml/h. The effluent is monitored at A₂₈₀ and the peak corresponding to 45-55 K daltons collected for a Fab fragment or 140-170K daltons for a whole antibody—this is the conjugate, which should be diluted to a working strength before use.

(ii) The preferred alternative procedure employs the use of an FPLC. The conjugate is purified on a Pharmacia Superdex 200 HR 10/30 column. 50 ml 0.007 g/ml solution of bovine serum albumin (BSA) is added to the conjugate (to bring the BSA concentration of the conjugate up to that of the elution buffer).

Before applying the sample, the column is equilibrated with two column volumes (50 ml) of elution buffer. The conjugate solution is then centrifuged at 10,000 g for 10 minutes to remove any particulate matter and applied to the FPLC column. The antibody is eluted from the column in the elution, and storage buffer at a flow rate of 0.5 ml/min. After the first 5 ml has passed through the column 0.5 ml fractions are collected.

The presence of antibody is detected though the use of an ultraviolet (UV) monitor and the fractions spanning the antibody peak are collected and analyzed for luminescent activity (normally fractions 16-21).

### Checking Luminescent Activity

The antibody fractions are diluted 1:500 in saline and 5 μl samples of each fraction spotted into the wells of an assay plate. The fractions are then tested for luminescent activity by reaction with activating reagents 1 and 2.-15 μl of activating reagent 1 is first added to the sample well, followed by 30 μl of activating reagent 2. This is normally achieved by automatic injectors in the luminometer, which is then activated to read the light emission from the well in question. The results are recorded using a repeat for each sample. Samples containing high levels of luminescent activity can then be confirmed in a microbial assay, in this example, a Salmonella Assay.

### Example 8

### AMPPD Use with Alkaline Phosphatase-Conjugated Anti-Salmonella Antibody

In order to generate a satisfactory luminescent signal, the antibody may be conjugated to the enzyme alkaline phosphatase and the substrate AMPPD employed in the immunoassay (AMPPD-3-(2′-spiroadamantane)-4-methoxy-4-(3″-phosphoryloxy)phenyl-1,2-dioxetane; 3-(4-methoxyspiro(1,2-dioxetane-3,2′-tricyclo(3.3.1.1(3,7))decan)-4-yl)phenyl phosphate). The diluent for this substrate is 0.9 g of CTAB (cetyltrimethyammonium bromide), 1.9 ml AMP (2-amino-2-methyl-1-propanol), 14.5 mg magnesium chloride.6H₂O, 1 mM, pH 9.6, in 100 ml distilled water.

### Reagents

### Wash Buffer

0.2M Tris (24.228 g/litre) 0.2M NaCl (11.688 g/litre)+0.05% (v/v) Tween (0.5 ml/litre).

Dissolve 24.228 g of Tris and 11.688 g of NaCl in 900 ml of dH20. Add 0.5 mls of Tween 20. Adjust the pH to 7.4 using HCl, make up to 1 litre and store at room temperature.

10× wash buffer concentrate (with preservative, sodium azide)

2M Tris (24.228 g/IOOml) 2M NaCl (11.688 g/IOOml)+0.5% Tween (0.5 mls/IOOml). Dissolve 24.228 g Tris and 11.688 g of NaCl in 80 mls distilled H₂0. Add 0.5 ml of Tween 20 and adjust the pH to 7.4 with conc. HCl. Make up to 100 ml with distilled H₂0 and store at room temp. To reconstitute the wash buffer, add 100 ml of concentrate to 900 ml of dH₂0 and store at room temperature.

### Elution and Storage Buffer

0.1M sodium phosphate buffer pH 6.3 with 0.15M NaCl 0.1% (w/v) bovine serum albumin (BSA) 0.05% NaN₃.

Make up a solution of 0.1M NaH₂PO₄ with 0.15M NaCl containing 0.1% w/v BSA (A) and 0.1M Na₂HPO₄ with 0.15M NaCl containing 0.1% w/v BSA (B). Add 100 ml of A to 50 ml B. Add 0.05% NaN₃, filter through a 0.22 mM filter and store at 4° C.

### Assay Buffer

0.01M NaH₂PO₄ (1.2 g/litre) 0.15M NaCl (8.75 g/litre) with 0.1% w/v NaN₃ and 0.25% w/v BSA.

Dissolve 1.2 g of NaH₂PO₄ and 8.75 g of NaCl in 900 ml dH₂0. Add 1.0 g NaN₃ and 2.5 g BSA. Allow to dissolve completely and adjust the pH to 7.4 with 1.0M NaOH. Make to 1000 ml with distilled H₂0. Filter through a 0.22 μM filter and store at 4° C.

### Detergent Solution

20% (w/v) SDS solution: Dissolve 5 g of SDS in 25 ml of dH₂0. Store at room temperature.

### Growth Enhancer

8 g sodium tetrathionate and 0.15 mg Brilliant Green added to 1 litre of sterile peptone broth. Mix gently until evenly distributed.

### Activating Reagent 1 (1 Litre)

6.3 ml of 70% nitric acid; 16.5 ml of 30% hydrogen peroxide; 977 ml of distilled water.

### Activating Reagent 2 (1 Litre)

10.0 g NaOH; 7.5 ml cetyltrimethyammoniumchloride; 983 ml distilled water

### Testing Fractions for Salmonella Binding

The wells of an assay plate are coated with standard concentrations of bacteria for 1 hour at 37° C. These standard concentrations are: 10⁶, 10⁵, 5×10⁴, 2.5×10⁴, 10⁴ and 5×10³ and blank wells containing 10⁶ E. coli. The fractions to be tested are diluted 1:100 in assay buffer and 50 ml is added to each well and incubated at 37° C. for 20 minutes. The wells are then read on the luminometer, as above. Those fractions demonstrating good binding in the assay are pooled and the optimal dilution for the pooled conjugate determined—normally 1:100 to 1:1000.

### Influence of Detergent on the Direct Binding Salmonella Assay

Novel black and white plate (Wallac) read on a tube luminometer (Berthold LB 9509) using the detergent, sodium dodecyl sulphate (SDS)

| | |
|---|---|
| 0% (w/v) SDS | 0.5% (w/v) SDS |

1:50 dilution of conjugate

| | | |
|---|---|---|
| Peptone | 1370 ± 127 | 1198 ± 112 |
| E. coli 10⁶ | 1039 ± 59 | 958 ± 242 |
| S. aberdeen 5 × 10³ | 1393 ± 130 | 1622 ± 21 |
| 10⁴ | 1423 ± 488 | 3199 ± 735 |
| 2.5 × 10⁴ | 1347 ± 152 | 6697 ± 1676 |
| 5 × 10⁴ | 1582 ± 333 | 12,231 ± 723 |
| 10⁵ | 2287 ± 248 | 22,245 ± 529 |
| 10⁶ | 16,860 ± 131 | 59,070 ± 1216 |

| | |
|---|---|
| 0% SDS | 0.5% SDS |

1:100 dilution of conjugate

| | | |
|---|---|---|
| Peptone | 1006 ± 203 | 974 ± 107 |
| 10⁶ E. coli | 1110 ± 127 | 922 ± 78 |
| S. aberdeen 5 × 10³ | 724 ± 24 | 1209 ± 66 |
| 10⁴ | 932 ± 231 | 1606 ± 243 |
| 2.5 × 10⁴ | 1094 ± 110 | 3933 ± 379 |
| 5 × 10⁴ | 919 ± 8 | 8721 ± 63 |
| 10⁵ | 1468 ± 121 | 15,009 ± 871 |
| 10⁶ | 11,109 ± 49 | 40,190 ± 783 |

1:150 dilution of conjugate

| | | |
|---|---|---|
| Peptone | 928 ± 47 | 998 ± 103 |
| 10⁶ E. coli | 659 ± 10 | 707 ± 11 |
| S. aberdeen 5 × 10³ | 622 ± 25 | 975 ± 42 |
| 10⁴ | 1104 ± 22 | 1415 ± 132 |
| 2.5 × 10⁴ | 1141 ± 22 | 1615 ± 132 |
| 5 × 10⁴ | 1035 ± 29 | 6576 ± 549 |
| 10⁵ | 1927 ± 220 | 12,817 ± 975 |
| 10⁶ | 11,872 ± 4303 | 31,571 ± 2 |

Results expressed as mean ± standard deviation; n = 7

### White Plate (Wallac), Read on Lucy I Plate Luminometer (1:100 Conjugate Dilution)

| | 0% (w/v) SDS | 0.1% (w/v) SDS | 0.2% (w/v) SDS |
|---|---|---|---|
| Peptone | 4,143 ± 107 | 4,288 ± 1.653 | 3,904 ± 59 |
| 10⁶ E. coli | 4,259 ± 209 | 4,151 ± 256 | 3,322 ± 479 |
| 10⁶ S. aberdeen | 35,532 ± 56,046 | 356,444 ± 102,877 | 365,496 ± 12,729 |
| 10⁵ S. aberdeen | 79,334 ± 1,248 | 143,796 ± 4,297 | 112,096 ± 3,841 |
| 5 × 10⁴ | 38,834 ± 2,624 | 51,909 ± 3,036 | 75,900 ± 5,798 |
| 2.5 × 10⁴ | 17,891 ± 3,422 | 28,622 ± 2,162 | 27,339 ± 2,635 |
| 10⁴ | 10,920 ± 305 | 11,324 ± 2,770 | 11,098 ± 828 |
| 5 × 10³ | 5,586 ± 327 | 8,536 ± 1,520 | 10,334 ± 2,264 |
| | 0.3% SDS | 0.4% SDS | 0.5% SDS |
| Peptone | 3,026 ± 473 | 3,499 ± 569 | 3,103 ± 267 |
| 10⁶ E. coli | 2,491 ± 107 | 3,197 ± 5 | 4,574 ± 1,885 |
| 10⁶ S. aberdeen | 352,714 ± 88,260 | 398,979 ± 44,871 | 374,007 ± 24,114 |
| 10⁵ S. aberdeen | 209,913 ± 40,150 | 199,287 ± 67,462 | 166,049 ± 288 |
| 5 × 10⁴ | 123,881 ± 15,994 | 120,481 ± 16,389 | 95,766 ± 6,186 |
| 2.5 × 10⁴ | 38,450 ± 1,411 | 59,399 ± 13,550 | 95,766 ± 6,186 |
| 10⁴ | 14,314 ± 617 | 29,127 ± 2,516 | 46,273 ± 2,310 |
| 5 × 10³ | 14,313 ± 2,881 | 25,175 ± 5,025 | 24,984 ± 1,727 |

### Effects of Various Levels of SDS on the Direct Binding Salmonella Assay (1:100 Conjugate Dilution) Using Black and White Plates

| | 0% SDS | 0.5% (w/v) SDS | 0.1% SDS |
|---|---|---|---|
| Peptone | 807 ± 24 | 705 ± 97 | 643 ± 54 |
| 10⁶ E. coli | 764 ± 399 | 684 ± 88 | 579 ± 37 |
| 10⁶ S. aberdeen | 5008 ± 639 | 16,959 ± 360 | 16,738 ± 1051 |
| 10⁵ S. aberdeen | 867 ± 100 | 2,259 ± 103 | 2,386 ± 263 |
| 5 × 10⁴ | 897 ± 102 | 1,826 ± 166 | 1,297 ± 4 |
| 2.5 × 10⁴ | 642 ± 90 | 1094 ± 156 | 1,277 ± 211 |
| 10⁴ | 927 ± 266 | 796 ± 23 | 1,662 ± 531 |
| 5 × 10³ | 891 ± 50 | 623 ± 9 | 591 ± 1 |
| | 0% SDS | 0.5% SDS | 0.1% SDS |
| Peptone | 643 ± 54 | 631 ± 38 | 503 ± 76 |
| 10⁶ E. coli | 579 ± 37 | 714 ± 74 | 658 ± 108 |
| 10⁶ S. aberdeen | 24,566 ± 4,287 | 26,621 ± 843 | 28,169 ± 1516 |
| 10⁵ S. aberdeen | 3,131 ± 121 | 3,947 ± 494 | 4,654 ± 636 |
| 5 × 10⁴ | 2,027 ± 206 | 2,213 ± 170 | 2321 ± 437 |
| 2.5 × 10⁴ | 1,617 ± 250 | 1,265 ± 46 | 1,442 ± 28 |
| 10⁴ | 1,102 ± 357 | 908 ± 10 | 930 ± 21 |
| 5 × 10³ | 575 ± 120 | 748 ± 4 | 678 ± 40 |

### Effects of Various Levels of TWEEN 20 on the Competitive Binding Salmonella Assay (1:100 Conjugate Dilution) Using Black and White Plates

| Competing | 0.5% | 0.5% | | | | |
|---|---|---|---|---|---|---|
| S. enteriditis | TWEEN | TWEEN | 1% TWEEN, | 1% TWEEN | 2% TWEEN | 2% TWEEN |
| (cfu/ml) | 2 min boil | 20 min boil | 2 min boil | 20 min boil | 2 min boil | 20 min boil |
| 10⁶ | 4274 ± 712 | 3531 ± 154 | 4497 ± 181 | 3216 ± 130 | 6747 ± 223 | 2074 ± 42 |
| 10⁵ | 37803 ± 2376 | 30413 ± 2985 | 48390 ± 2811 | 25614 ± 1030 | 53067 ± 701 | 6619 ± 573 |
| 10⁴ | 200745 ± 12074 | 190752 ± 6265 | 222178 ± 3436 | 171024 ± 2620 | 226168 ± 24620 | 63938 ± 4766 |
| 10³ | 277066 ± 20343 | 305744 ± 27327 | 305343 ± 15168 | 270782 ± 15522 | 266820 ± 24059 | 281305 ± 20480 |
| 0 | 370245 ± 16595 | 370245 ± 16595 | 370245 ± 16595 | 370245 ± 16595 | 370245 ± 16595 | 370245 ± 16595 |

(Mean ± standard deviation, relative light units)

Effects of Anti Salmonella mAB Incubation Times with 1:100 Anti 2b Conjugate

Monoclonal antibody (1:100 dilution) was incubated with either Salmonella or Listeria to determine optimum incubation times:

| | 30 mins | 40 mins | 60 mins |
|---|---|---|---|
| 10⁶ S. aberdeen | 598080 | 609383 | 593741 |
| 10⁵ S. aberdeen | 716821 | 644854 | 629340 |
| 10⁴ S. aberdeen | 276239 | 328483 | 371163 |
| 10³ S. aberdeen | 20117 | 19454 | 29194 |
| 10² S. aberdeen | 5439 | 7204 | 17242 |
| 10⁶ L. innocua | 6376 | 8909 | 12151 |

(Read on Lucy I Plate Luminometer, results shown in relative light units)

Effects of Anti Salmonella mAB Incubation Times with 1:200 Anti 2b Conjugate

Monoclonal antibody (1:200 dilution) was incubated with either Salmonella or Listeria to determine optimum incubation times:

| | 30 mins | 40 mins | 60 mins |
|---|---|---|---|
| 10⁶ S. aberdeen | 339224 | 340594 | 356500 |
| 10⁵ S. aberdeen | 344236 | 353391 | 372633 |
| 10⁴ S. aberdeen | 247657 | 243787 | 256889 |
| 10³ S. aberdeen | 14023 | 16848 | 22829 |
| 10² S. aberdeen | 4869 | 6846 | 10817 |
| 10⁶ L. innocua | 11395 | 7775 | 11455 |

(Read on Lucy I Plate Luminometer, results shown in relative light units)

### Effects of SDS Concentration on Food Cultures

White Plate read on Lucy 1 luminometer

| −ve Chicken | | +ve Chicken* | |
|---|---|---|---|
| 0% SDS | 1755 ± 272 | 0% SDS | 47,872 ± 4509 |
| 0.1% SDS | 3426 ± 316 | 0.1% SDS | 131,488 ± 15,357 |
| 0.5% SDS | 4494 ± 904 | 0.5% SDS | 36,770 ± 2,020 |

| −ve Mayonnaise | | +ve Mayonnaise* | |
|---|---|---|---|
| 0% SDS | 1896 ± 163 | 0% SDS | 255,232 ± 26,535 |
| 0.1% SDS | 4180 ± 610 | 0.1% SDS | 746,670 ± 86,449 |
| 0.5% SDS | 3260 ± 733 | 0.5% SDS | 387,924 ± 106,504 |

| −ve Drinking | | +ve Drinking | |
|---|---|---|---|
| Chocolate | | Chocolate* | |
| 0% SDS | 2047 ± 134 | 0% SDS | 4051 ± 136 |
| 0.1% SDS | 1315 ± 63 | 0.1% SDS | 45,626 ± 3204 |
| 0.5% SDS | 3266 ± 142 | 0.5% SDS | 110,756 ± 5737 |

*All +ve (bacteria positive) food cultures were contaminated with 10 C.F.U. of S. aberdeen and cultured for 18 hours (25 g in 225 ml) of Peptone broth +8 g/1 sodium tetrathionate and 0.15 mg/l of Brilliant Green. The bacteria negative cultures (−ve) were contaminated and also cultured for 18 hours. 5 ml samples of all the food cultures (with or without SDS) were heated for 20 minutes in a boiling water bath prior to the assay.

### Effect of Different Detergents on Release and Detection of Core Oligosaccharide

| | | | | | | | | NO |
|---|---|---|---|---|---|---|---|---|
| | | | | | | | | DETERGENT |
| | | | TRITON | TWEEN | SODIUM | | | BOILED |
| | CTAB | CTAC | X-100 | 20 | CHOLATE | 0.1% SDS | 0.5% SDS | ONLY |
| 10⁶ | 866920 | 881712 | 455886 | 1025524 | 917476 | 895811 | 884352 | 36939 |
| 10⁵ | 775871 | 792108 | 867745 | 573381 | 529553 | 717259 | 959119 | 19373 |
| 10⁴ | 203215 | 205093 | 203602 | 16893 | 67710 | 65960 | 495603 | 9173 |
| 10³ | 21541 | 22758 | 22570 | 9594 | 12088 | 12375 | 30399 | 7862 |
| 10² | 9746 | 12485 | 10494 | 8355 | 8921 | 9525 | 9987 | 9109 |
| 10¹ | 8901 | 10321 | 9981 | 8117 | 8858 | 10248 | 9918 | 9268 |
| 10⁶ L. innocua | 7593 | 7892 | 7076 | 7226 | 7495 | 7863 | 8051 | 8454 |

(Mean of three separate experiments, relative light units)

SDS provides the most reliable and reproducible results for dissolution of food sample-based Salmonella LPS into monomers. However only the Tween can be used for this purpose in the competitive assay due to protein-detergent interactions with the other detergents.

### Effects of Varying Anti-Salmonella Antibody Levels on Detection of Salmonella in the Competitive Assay.

| (cfu/ml) | 50 ng/ml | 25 ng/ml | 10 ng/ml | 5 ng/ml |
|---|---|---|---|---|
| 10⁶ | 9490 | 7828 | 8676 | 8198 |
| 10⁵ | 23588 | 19974 | 14138 | 11554 |
| 10⁴ | 91779 | 70822 | 32551 | 23628 |
| 10³ | 149420 | 82361 | 42485 | 31153 |
| 10² | 151012 | 99182 | 55797 | 35750 |
| 10¹ | 151187 | 109799 | 71297 | 40671 |
| No competing | 159874 | 125724 | 86091 | 45119 |
| bacteria | | | | |

(mean of three separate experiments, relative light units)

### REFERENCES

Buzby, J. C. and Roberts, T. (1997) Economic costs and trade impacts of microbial foodborne illness. World Health Stat. Q. 50 (1-2):57-66.
Chau, P. Y. and Leung, Y. K. (2008) Inhibitory action of various plating media on the growth of certain Salmonella serotypes. J. App. Microbiol. 45 (3):341-345.
King, L. (2009) Salmonella Rapid detection interagency group meeting. FDA executive summary, 30 Jan. 2009.
Lee, W. C., Lee, M. J., Kim, J. S. and Park, S. Y. (2001) Foodborne illness outbreaks in Korea and Japan studied retrospectively. J Food Prot 64:899-902.
Meade, P. S., Slutsker, L., Dietz, V., McCaig, L. F., Bresee, J. S., Shapiro, C., Griffin, P. M. and Tauxe, R. V. (1999) Food related illness and death in the United States. Emerg. Infect. Dis. 5:607-625.
Qualtiere, L. F., Anderson, A. G. and Meyers, P. (1977) Effects of ionic and non-ionic detergents on antigen-antibody reactions. J. Immunol. 119 (5):1645-1651.
Raetz, C. R. H. (1996) Bacterial lipopolysaccharides: a remarkable family of bioactive macroamphiphiles, in Escherichia coli and Salmonella, Vol. I (Neidhardt, F., ed.), Second Edition, pp. 1035-1063, ASM Publications, Washington, D.C.
Reissbrodt, R., Rienaecker, I., Romanova, J. M., Freestone, P. P. E., Haigh, R. D., Lyte, M., Tschäpe, H. and Williams, P. H. (2002) Resuscitation of Salmonella enterica serovar Typhimurium and enterohemorrhagic Escherichia coli from the viable but nonculturable state by heat stable enterobacterial autoinducer. App. Env. Microbiol. 68 (10):4788-4794.
Stephens, P. J., Druggan, P., Nebe-von Caron, G. (2000) Stressed Salmonella are exposed to reactive oxygen species from two independent sources during recovery in conventional culture media. Int. J. Food Microbiol. 60:269-285.

1. A culture medium for the growth of at least one microorganism consisting essentially of:
(i) A base broth;
(ii) At least one growth inhibitor selected from the group consisting of brilliant green, nalidixic acid and lithium chloride; and
(iii) Optionally, at least one growth promoter selected from the group consisting of sodium tetrathionate, ammonium ferric citrate and sodium citrate.

2. A culture medium as claimed in claim 1 wherein the growth inhibitor is brilliant green in an amount of between about 0.05 to about 0.25 mg/L.

3. A culture medium as claimed in claim 1 wherein the growth inhibitors are nalidixic acid in an amount of between about 1 to 3 mg/L and lithium chloride in an amount of between about 1 to about 3 g/L

4. A culture medium as claimed in claim 1 which comprises a growth promoter, wherein the growth promoter is sodium tetrathionate in an amount of between about 4 to about 12 g/L.

5. A culture medium as claimed in claim 1 which comprises a growth promoter, wherein the growth promoter is ammonium ferric citrate in an amount of between about 200 to 300 mg/L.

6. A culture medium as claimed in claim 7 further comprising the growth promoter sodium citrate in an amount of between about 10 to 20 g/L, more particularly about 15 g/L.

7. A culture medium as claimed in claim 1 wherein the at least one microorganism is a salmonella spp.

8. A culture medium as claimed in claim 1 wherein the at least one microorganism is a shigella spp.

9. A culture medium as claimed in claim 1 wherein the at least one microorganism is a Listeria spp.

10. An assay method for detecting the presence or absence of a microorganism of interest in a test sample, the method comprising:
(i) Culturing the test sample in a culture medium which allows for propagation of the microorganism of interest;
(ii) Treating the test sample sufficient to release one or more core oligosaccharides from any microorganisms present within the test sample;
(iii) Exposing the test sample to at least one binding member which has binding specificity to a core oligosaccharide of the microorganism of interest; and
(iv) Detecting any binding of the at least one binding member to a core oligosaccharide of the microorganism of interest.

11. The method of claim 10 wherein step (ii) comprises:
(a) adding a detergent to the test sample containing said microorganism of interest to provide a detergent-culture solution; and
(b) heating the detergent-culture solution to a temperature sufficient to release the core oligosaccharide.

12. The method according to claim 11 wherein the detergent is sodium dodecyl sulphate, TWEEN 20, TWEEN 40, TWEEN 60 or TWEEN 80.

13. The method of claim 10 wherein step (i) is performed using a culture medium for the growth of at least one microorganism consisting essentially of a base broth and at least one growth inhibitor selected from the group consisting of brilliant green, nalidixic acid and lithium chloride.

14. The method of claim 10 wherein step (iv) is by detection of a luminescent signal.

15. The method of claim 14 wherein the luminescent signal is produced by an acridinium ester.

16. A method of releasing the core oligosaccharide from the cell of a microorganism comprising:
(i) adding a detergent to at least one culture sample containing said microorganism to provide a detergent-culture solution; and
(ii) heating the detergent-culture solution to a temperature sufficient to release the core oligosaccharide.

17. A method according to claim 16 wherein the detergent is sodium dodecyl sulphate, TWEEN 20, TWEEN 40, TWEEN 60 or TWEEN 80.

18. (canceled)

19. A method of specific detection of a microorganism selected from the group consisting of salmonella, shigella and listeria, comprising contacting a binding member which has binding specifically to a core oligosaccharide.

20. A method of growing at least one bacteria, particularly salmonella, shigella or hysteria, in a culture medium according to claim 1.

21. The method of claim 10 wherein the core oligosaccharide epitope is: [chemical structure]
